(19) United States
(12) Patent Application Publication
(10) Pub. No.: US 2014/0371103 A1
(43) Pub. Date: Dec. 18, 2014

(54) METHODS AND REAGENTS FOR EVALUATING AUTOIMMUNE DISEASE AND DETERMINING ANTIBODY REPERTOIRE

(71) Applicant: DIOGENIX, INC., Gaithersburg, MD (US)

(72) Inventors: Eric M. Eastman, Highland, MD (US); Douglas W. Bigwood, Madison, CT (US)

(21) Appl. No.: 14/475,007

(22) Filed: Sep. 2, 2014

Related U.S. Application Data
(63) Continuation-in-part of application No. PCT/US2013/028803, filed on Mar. 4, 2013.
(60) Provisional application No. 61/606,124, filed on Mar. 2, 2012.

(51) Int. Cl. C12Q 1/68; G01N 33/68

(52) U.S. Cl. CPC C12Q 1/6883; G01N 33/6893; G01N 2500/04; C12Q 2600/106; C12Q 2600/158
USPC 506/9; 435/6.11; 530/300

(57) ABSTRACT

The invention provides methods and kits for determining an antibody or T-cell receptor repertoire in a sample containing B-cells and/or T-cells, and provides a method for evaluating a patient for the presence of an autoimmune disorder.

### PRIORITY

This application is a continuation-in-part of International Application No. PCT/U.S. 2013/028803, filed Mar. 4, 2013, which claims priority to and the benefit of U.S. Provisional Application No. 61/606,124, filed Mar. 2, 2012, both of which are hereby incorporated by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: DIOG_{—}005_{—}01US_SeqList_ST25.txt, date recorded: Sep. 2, 2014, file size 28 kilobytes).

### BACKGROUND

The underlying cause of many autoimmune disorders, such as Multiple Sclerosis (MS), is not well-understood. For example, MS includes inflammatory, autoimmune and neurodegenerative processes. Although T cells have long been recognized as key players in the immunopathology of MS, there is mounting evidence that B cells also play an important role in the disease process by participating in antigen presentation, T cell activation and the production of antibodies (Abs) against self-antigen (Ag) (1-6). The infiltration and colonization of B cells in the Central Nervous System (CNS) of MS patients leads to the persistent synthesis of Ab, including Ab against both myelin and non-myelin Ags (3, 5). Auto-Ab against components of the myelin sheath that surrounds and protects neurons in the CNS may be directly involved in the demyelination process, leading to irreparable nerve damage and permanent physical disability (7, 8).

The current standard of care used to evaluate patients with unexplained neurological dysfunction includes testing their cerebrospinal fluid (CSF) to evaluate the status of inflammatory and auto-immune processes in their CNS. Proteins in CSF and serum from the same patient are analyzed in parallel by gel electrophoresis and/or isoelectric focusing (IEF) to look for the presence of oligoclonal bands (OCB). The presence of immunoglobulin protein bands in the CSF that are not detected in serum is evidence of localized inflammation and/or auto-immune activity in the CNS, as compared to a more systemic reaction. Although the OCB test has reasonable sensitivity, it has very low specificity (8, 9), since OCBs are detectable in patients with many other diseases (5, 9). See, in particular, Table 1 of Awad et al., Analyses of cerebrospinal fluid in the diagnosis and monitoring of multiple sclerosis. J. Neuroimmunol. 219(1-2):1-7(2010).

Notably however, certain disease-associated antibodies, e.g. anti-myelin Ab, are detectable in the CSF of approximately 28% of MS and CIS patients who do not have detectable OCB (8), suggesting that the OCB test misses some patients with Ab-producing B cells in their CNS. A more comprehensive method of measuring Ab or T-cell receptor diversity may provide (i) greater diagnostic assay sensitivity and specificity, for MS and other autoimmune conditions and (ii) a biological baseline from which to better understand the progression of autoimmune diseases, including MS.

### SUMMARY OF THE INVENTION

In one aspect, the invention provides methods and kits for determining an antibody or T-cell receptor repertoire in a sample containing B-cells and/or T-cells. The method comprises amplifying target polynucleotide sequences that encode an antigen binding region of an antibody gene family or T-cell receptor gene family using a set of non-degenerate primers, and determining the amplified sequences. In various embodiments, the sequences are clonally amplified, and a plurality of sequences generated. The set of non-degenerate primers comprises at least one primer specific for amplifying each member of a family of related sequences. For example, where the target sequence is the variable region of rearranged immunoglobulin heavy chain genes (IGHV), the non-degenerate primers amplify substantially each IGHV family sequence.

In another aspect, the invention provides a method for evaluating a patient for the presence of an autoimmune disorder. The method according to this aspect comprises determining amino acid substitutions in the IGH-VDJ region of an antibody gene family from a plurality of immune cells from the patient; and classifying the patient as having or in the early-stages of developing an autoimmune disorder.

In another aspect, the invention provides a method for making an agent for treating autoimmunity. The method according to this aspect comprises determining the variable sequence of an autoreactive antibody, optionally in accordance with the methods and kits described herein, identifying an epitope to which said autoreactive antibody binds, and synthesizing a peptide comprising said epitope to thereby make an agent for treating autoimmunity.

Various other aspects and embodiments will be apparent from the following detailed description and claims.

### DESCRIPTION OF THE FIGURES

FIG. 1 illustrates Ig heavy chain rearrangement.

FIG. 2 illustrates somatic hypermutation within the Ig heavy chain.

FIG. 3 shows an alignment of the unique sequences at the 5′ end of the human IGHV4 family (including subfamilies) gene segments (SEQ ID NOS:50-67).

FIG. 4 is a summary of all unique 5′ (Forward) PCR primer sequences along with the % of total sequences represented by each primer sequence (SEQ ID NOS:68-73).

FIG. 5 is an alignment of all the unique sequences at the 3′ end of the human IGHJ gene segments (SEQ ID NOS:74-86).

FIG. 6 is a summary of all unique 3′ (Reverse) PCR primer sequences based on the example illustrated in FIG. 5, along with the % of total sequences represented by each primer sequence (SEQ ID NOS:87-93).

FIG. 7 shows an alignment of all IGHV4 sequences for the region including aa1-aa19 (n=17) (SEQ ID NOS:94-170).

FIG. 8 shows a comparison of specific amino acid changes and the percent of all NGS sequences that express each change for a short region (aa 53-57) of the IGHV-D-J (VH4) region across four cohorts.

FIG. 9 shows a comparison of specific amino acid changes for almost the entire IGHV-D-J (VH4) region (aa 31-91) across four cohorts.

### DETAILED DESCRIPTION OF THE INVENTION

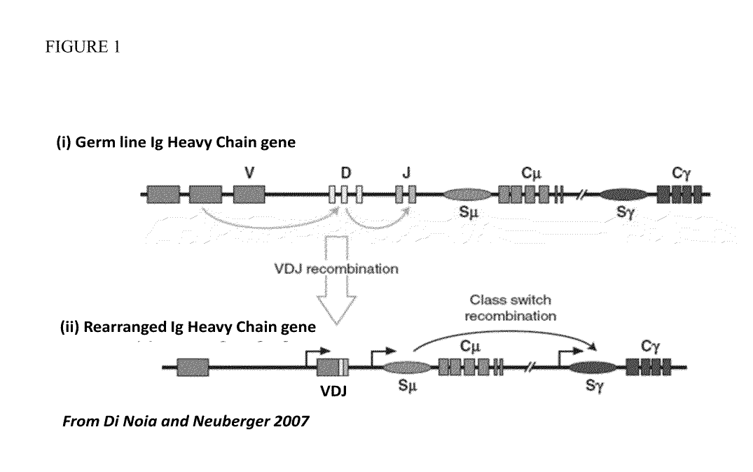

In naïve B cells, the initial level of antibody (Ab) diversity is generated by a complex series of DNA rearrangements in the immunoglobulin (Ig) gene locus (10). See FIG. 1. Antigen recognition (foreign or self) is primarily determined by antigen-specific receptor proteins presented on the surface of T cells (“T cell receptors”—TCRs), B cells (“B cell receptors”—BCRs), and antibodies that are secreted by B cells and B cell-derived plasma cells. Amazingly, the mammalian immune system can produce Ag-binding receptor proteins capable of recognizing virtually any molecule, whether natural or man-made. This is possible due to the tremendous complexity of the genes that code for these receptor proteins and processes exponentially expand this complexity.

Abs are made up of different combinations of longer (heavy chain) and shorter (light chain) proteins. Different types of Ab molecules contain different numbers of heavy and light chains but the primary functional unit includes four protein molecules, two heavy chain and two light chain molecules that assemble to form the classic Y shape. Abs of the

IgG type contain one of these functional units (4 protein molecules), while IgM Abs contain 5 units (20 protein molecules). Each B cell produces only one heavy and one light chain protein molecule such that all the Ab molecules produced by a single B cell are identical. The specificity of each antibody molecule is determined by the specific amino acid sequences in defined “variable” regions at the amino terminal ends of the heavy and light chains that combine to form the Ag binding domain of the antibody molecules. The variable regions that make up the Ag binding domain are broken up into two different types of functional domains. Framework regions (FR), which are primarily responsible for determining the three dimensional structure of the Ag binding domain, are interspersed with complementarity determining regions (CDR), which contain the amino acid residues primarily responsible for direct interaction (contact points) with the Ag itself. The diversity in Ag binding specificity comes from a combination of DNA rearrangements and somatic mutations.

Initial Ab diversity is determined by the complex rearrangement of a series of genes or gene segments contained in three different Ig gene loci in the human genome, one for the Ig heavy (IGH) chain and one for each of two types of Ig light (IGL) chains (kappa and lambda). Each locus is located on a different chromosome and contains multiple copies of either 2 or 3 different types (“variable”, “diversity” and “joining”) of gene segments. The IGH locus contains all 3 types of gene segments and the IGL loci contain only variable and joining gene segments. By way of example, consider the human immunoglobulin heavy (IGH) chain locus that produces the Ig heavy chain protein. This locus is located at the telomeric end of human chromosome 14 and spans almost 1.0 million base pairs (bp). The IGH gene locus contains at least 200 different variable (IGHV) gene segments, however many of these are pseudogenes or otherwise non-functional. It is estimated that there are 51 functional IGHV gene segments grouped into 7 families (VH1-VH7) and each IGHV family contains a number of sub-families, based on DNA sequence differences (11, 12). Additionally, there are 27 heavy chain diversity (IGHD) gene segments and 6 joining (IGHJ) gene segments, each having a unique sequence (11, 12). Numerous polymorphic alleles also have been identified for many of these gene segments.

During B cell differentiation, a complex series of rearrangements occurs to align one copy of each type of gene segments in the IGH and IHL loci. In the IGH gene locus, one IGHV segment is moved adjacent to one of the IGHD segments and one of the IGHJ segments to produce a unique VDJ rearrangement. Since recombination is essentially random and any IGHV gene can recombine with any IGHD and IGHJ gene, there is the potential to generate at least 8,262 (51×27×6) possible functional combinations. In reality, however, the potential number of rearranged sequences is much, much higher due to additional DNA modifications (sequence extensions and deletions) that occur during the rearrangement process, particularly at the D-J junction. Similar rearrangements of the IGLV and IGLJ genes segments occur to generate many different functional light chains.

In any given B-cell, any recombined heavy chain can combine with any recombined light chain. It is estimated that the total number of different Ab molecules that can be generated is 10⁵-10⁷ (10).

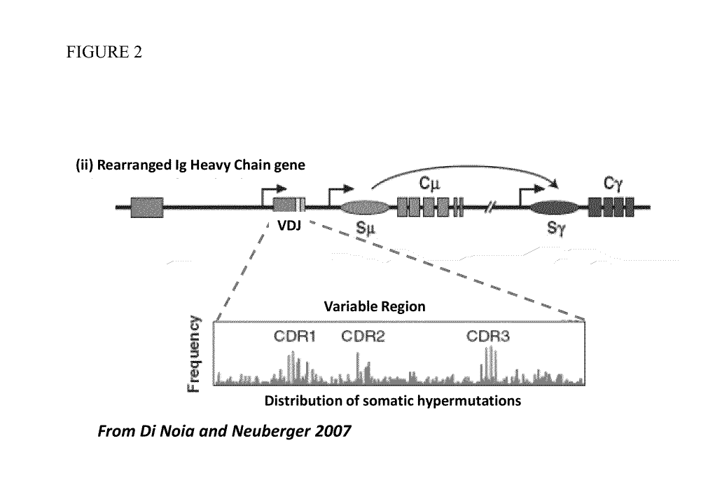

However, these initial Abs tend to have relatively low Ag binding affinities. With repeated Ag exposure, B cells become activated and proliferate. During this process, the “variable” regions, including the V, D and J gene segments of rearranged Ig heavy chain genes and the V and J gene segments of rearranged light chain genes, undergo somatic hypermutation to increase the specificity and affinity of Abs for their cognate Ag—a process called “affinity maturation” (10, 13). By definition, this leads to greatly increased sequence diversity of Ab genes, resulting in the production of billions of unique Ab proteins. The amino acids in the CDRs are strategically more affected by the hypermutation process than the FRs (see FIG. 2).

Ultimately, the antigen specificity of each antibody molecule is determined by the specific amino acid sequences of the IGH-VDJ and IGL-VJ segments. Minor amino acid changes in these regions can significantly alter antigen specificity and the affinity of the Ab for the cognate Ag.

Generating a comprehensive B cell repertoire requires that the DNA sequence of a substantial number of the unique rearranged IGH-VDJ and IGL-VJ gene segments are accurately determined. Since most of the Ag binding specificity is determined by the Ig heavy chain, primarily due to the extensive diversity of the IGH-CDR3 region (14), in preferred embodiments the invention comprises analysis of IGH-VDJ sequences. Similar approaches are used to analyze light chain diversity in B cells and TCRs in T cells.

Since Ab diversity is determined by sequence diversity across the entire IGH-VDJ region, sequences for this entire region must be generated and analyzed. This can be done by first selectively amplifying, using polymerase chain reaction (PCR), the rearranged

IGH-VDJ region from genomic DNA (gDNA) or expressed messenger RNA (mRNA) by first converting the mRNA to complementary DNA (cDNA). Once amplified, various methods that are available to generate DNA sequence from the resulting PCR products. The key to generating a comprehensive B cell Ig heavy chain repertoire for an individual is generating sufficient highly accurate sequences for each IGH-VDJ region in a particular B cell population. This requires using an amplification protocol capable of efficiently and accurately amplifying each and every possible VDJ recombination with minimal bias. The challenge is to design a combination of forward (5′) and reverse (3′) PCR primers capable of achieving this objective.

Due to the extensive sequence diversity across the entire VDJ region that results from the large number of individual germline IGHV, IGHD and IGHJ gene segments plus somatic hypermutation, there are no regions of conserved or invariant sequence long enough to design unique PCR primers. As a result, many published reports of methods to generate B cell repertoires use pairs (forward and reverse) of degenerate PCR primers (1, 6, 15-17). A “degenerate primer” is actually a mixture of oligonucleotides with different sequences and should contain all the individual oligonucleotides required to anneal to all the target sequences in a complex mixture, such as DNA from a population of B cells. Typically, degenerate primers are designed by specifying two or more (up to 4) nucleotides at specific sites in the oligonucleotide sequence where the target sequence is ambiguous or where there are known to be sequence polymorphisms. Thus, a degenerate primer with 2 alternative nucleotides at a single position will actually contain 2 different oligonucleotides, each differing by one nucleotide. A degenerate primer with 3 alternative nucleotides at a single position will contain 3 different oligonucleotides, each differing by one nucleotide. A degenerate primer with 2 alternative nucleotides at two different nucleotide positions will contain 4 (2×2) different oligonucleotides each with a unique sequence. A degenerate primer with 2 alternative nucleotides at one position and 4 alternative nucleotides at a different position will contain 8 (2×4) different oligonucleotides each with a unique sequence and so forth.

The advantage of this approach is that multiple oligonucleotides, each with a slightly different sequence can be synthesized in a single DNA synthesis reaction rather than having to synthesize many individual primers. This approach reduces the cost of primer synthesis and the complexity of the PCR amplification process. In practice, however, the resulting degenerate primer often contains individual sequences that are not specific for the target, e.g. the rearranged VDJ region. These sequences dilute the concentration of target-specific sequences in the PCR reaction and may lead to reduced amplification efficiency and/or primer limitation. Furthermore, these non-target-specific primers may actually anneal to other sequences in the genome leading to the generation of undesired PCR products and, ultimately, undesired sequences that introduce noise that can complicate data analysis.

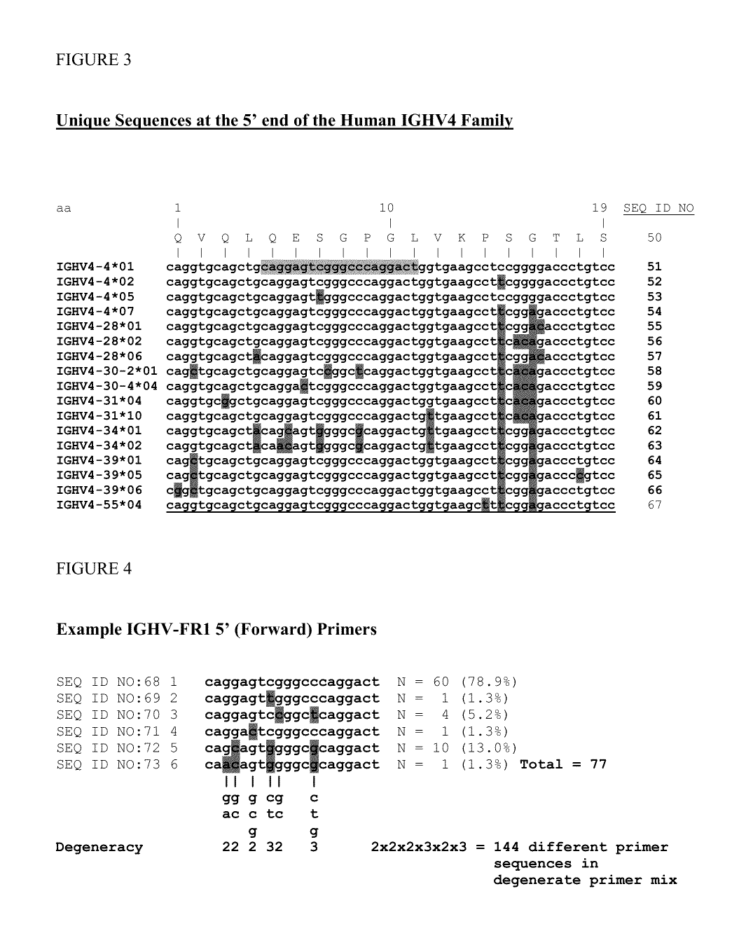

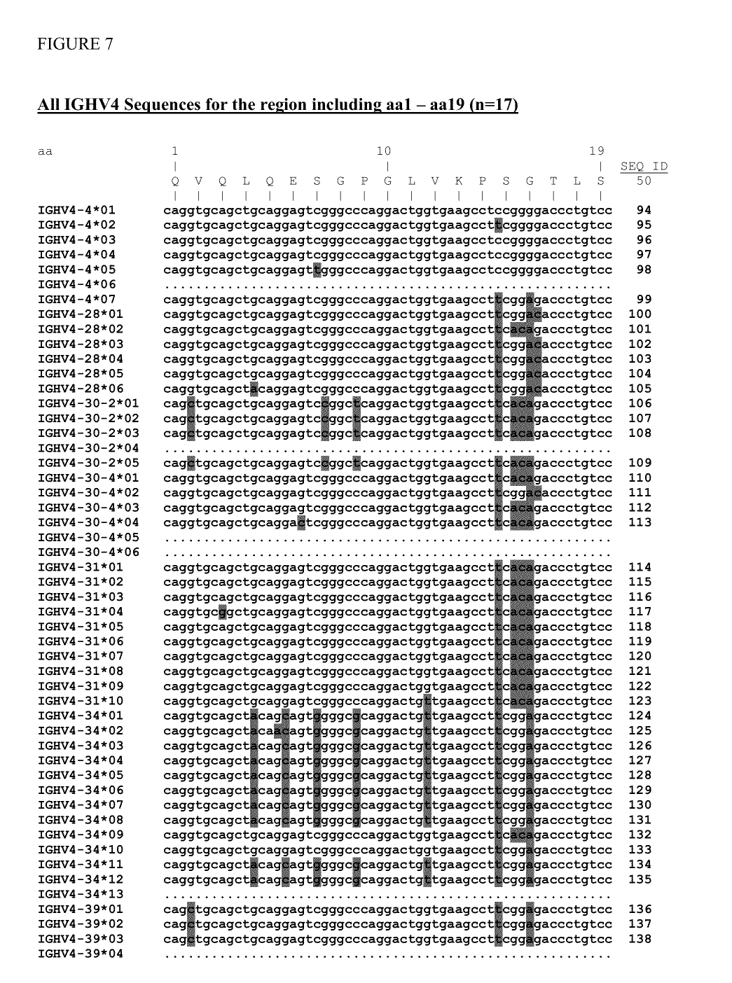

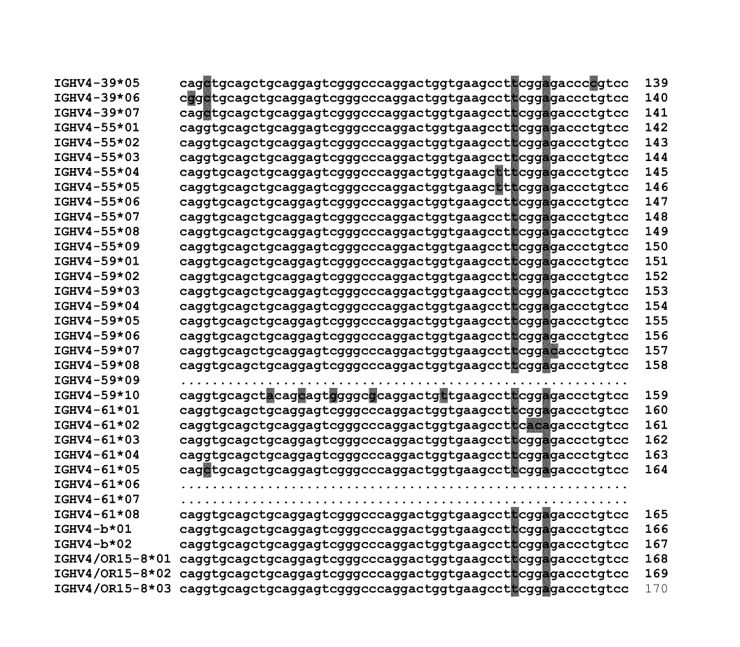

By way of example, FIG. 3 shows an alignment of the unique sequences at the 5′ end of the human IGHV4 family (including subfamilies) gene segments extracted from the September 2011 version of the ImMunoGeneTics (IMGT) database (12) (see FIG. 7). An alignment of the 5′ ends of all IGHV4-related gene sequences is included in Example 1. Many of the individual sequences in this region are very similar or identical. These sequences include the codons that code for the first 19 amino acids (aa) of the variable region segment after the leader sequence. This is in the first Framework region (FR1) of IGHV. A baseline sequence for IGHV4-4*01 (IGHV4 family; sub-family 04; allele *01) is displayed as the first nucleotide sequence along with the corresponding amino acid sequence. For the other IGHV4 family/subfamily members, the nucleotides that differ from the baseline sequence are highlighted. An example 5′ (Forward) PCR primer sequence is highlighted in IGHV4-4*01 sequence. This primer sequence was chosen since it is a fairly highly conserved region of the IGHV4 FR1 segment. This is only one example. Primers could be designed anywhere within this region, the upstream leader sequences or downstream of this region depending on the region to be amplified and the specific application.

Detailed analysis of the sequences contained in the IMGT database reveals that the earlier methods and primer sequences used to generate B cell repertoires are inadequate to generate a complete IGHV repertoire.

FIG. 4 is a summary of all unique 5′ (Forward) PCR primer sequences based on the example presented above along with the % of total sequences represented by each primer sequence. The overall primer degeneracy that would be generated if a degenerate primer set is used is summarized below the sequences.

The first thing to notice is that synthesis of a single degenerate primer capable of covering all members of the human IGHV4 family for this region would yield a mixture of 144 different IGHV 5′ primers. However, it can be seen that only 6 primer sequences are actually required to cover all IGHV4 subfamilies. These 6 primers could be used individually in separate PCR reactions or pooled to allow amplification of all IGHV4 subfamily members in a single PCR reaction. The synthesis of 6 individual non-degenerate primers provides complete coverage and does not generate primers with sequences that do not match any of the IGHV4 subfamily sequences. Another way of looking at the issue is that the mixture of degenerate primers contains 138 (144−6=138) primers that do not have a 100% match with any of the IGHV4 sequences in this region. This represents approximately 96% of the primers in the degenerate primer mixture. Although the design and synthesis of multiple degenerate primers to cover this region could reduce the degeneracy required in a single primer set, this approach would still generate many more primer sequences than are actually required.

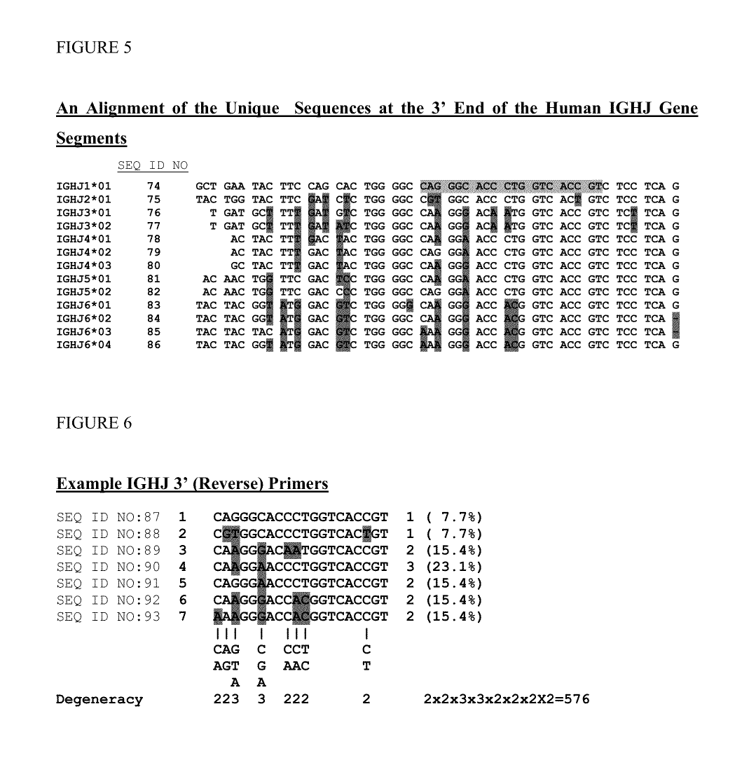

FIG. 5 is an alignment of all the unique sequences at the 3′ end of the human IGHJ gene segments (including alleles) extracted from the September 2011 version of the IMGT database. A baseline sequence for IGHJ1*01 (IGHJ1 family; allele *01) is displayed as the first nucleotide sequence. For the other IGHJ family members, the nucleotides that differ from the baseline sequence are highlighted. An example 3′ (Reverse) PCR primer sequence is highlighted in the IGHJ1*01 sequence. Note that this sequence will need to be reverse-complemented to generate the actual sequence used to synthesize reverse primers. This primer sequence was chosen since it is in a fairly highly conserved region of the IGHJ gene segment. This is only one example. Primers could be designed anywhere within this region or downstream of this region depending on the region to be amplified and the specific application.

FIG. 6 is a summary of all unique 3′ (Reverse) PCR primer sequences based on the example presented above along with the % of total sequences represented by each primer sequence. The overall primer degeneracy that would be generated if a degenerate primer set is used is summarized below the sequences.

The synthesis of a single degenerate primer capable of covering all members of the human IGHJ family for this region would yield a mixture of 576 different IGHJ 3′ primers. However, it can be seen that only 7 primer sequences are actually required to cover all IGHJ families and alleles. These 7 primers could be used individually in separate PCR reactions or pooled to allow amplification of all IGHJ family members in a single PCR reaction. The synthesis of 7 individual non-degenerate primers provides complete coverage and does not generate primers with sequences that do not match any of the IGHJ sequences. Another way of looking at the issue is that the mixture of degenerate primers contains 569 (576−7=569) primers that do not have a 100% match with any of the IGHJ sequences in this region. This represents almost 99% of the primers in the degenerate primer mixture. Although the design and synthesis of multiple degenerate primers to cover this region could reduce the degeneracy required in a single primer set, this approach would still generate many more primer sequences than are actually required.

Although the above examples only discuss the design of one set of 5′ (Forward) and 3′ (Reverse) primers for PCR amplification of rearranged human IGH-V4DJ gene segments, this approach can be used to design non-degenerate forward and reverse PCR primers to amplify the other 6 IGHV families and for the T cell receptor (TCR) families. TCRs are antibody-like recognition/signaling molecules that reside on the surface of T cells. As for the B cell-produced IGH families, there are also a number of distinct TCR families with extensive sequence diversity. Similar methods, requiring degenerate PCR primers, are used to generate T cell repertoires. In addition, nested sets of non-degenerate PCR primers could be designed to increase amplification sensitivity and specificity under certain circumstances.

In certain aspects, the invention provides a set of non-degenerate primers for amplifying, for example, the human B cell repertoire rather than degenerate primers that contain numerous primer sequences that are not specific for the target sequence. This approach should improve PCR efficiency and yield, and reduce amplification of undesired PCR products. Exemplary sets of nested 5′ (Forward) and 3′ (Reverse) PCR primers for amplifying rearranged IGH-V4DJ region are shown in Tables 1-3.

Alternatively, pairs of individual non-degenerate primers could be used to further increase PCR efficiency. In this case, all combinations of forward and reverse primers would be used in individual PCR reactions and analyzed separately or pooled and analyzed together. These embodiments can be performed with microfluidic arrays or in microdroplet-based approaches that can process thousands of PCR reactions simultaneously. For example, the Access Array™ system developed by Fluidigm can simultaneously process 2,304 individual PCR reactions starting with 48 samples and 48 primer pairs. In this example, 48 PCR reactions (48 primer pairs) are processed for each of the 48 samples (48×48=2,304). The 48 individual PCR reactions for each sample are then automatically pooled to yield 48 samples containing 48 PCR products.

By way of example, all rearranged IGH-V4DJ gene segments could be amplified using 42 (6 forward×7 reverse) primer pairs. This is compatible with the Fluidigm's standard Access Array™ format.

Pooled PCR products generated using either of the approaches described above would be appropriate templates for “deep” Next Generation DNA Sequencing.

The individual primers do not have to contain the entire length of the “Primer Regions” identified in the examples above. The Primer Regions identify the general regions of least variability which represent candidate regions for primer design.

Thus, in various embodiments, the invention provides a method for determining a repertoire of antibodies or T-cell receptor(s) in a patient. The method comprises amplifying target polynucleotides from a sample containing polynucleotides from B-cells or T-cells, the target polynucleotides encoding an antigen-binding region of an antibody gene family or T-cell receptor gene family, and said amplifying being conducted with a set of non-degenerate primers. The set of non-degenerate primers comprises at least one primer specific for amplifying each member of the family or sub-family. The method further comprises determining the sequences in the sample. For example, in some embodiments the polynucleotides are from B-cells, and the non-degenerate primers are specific for a variable region of an immunoglobulin gene or segment thereof.

In some embodiments, the set of primers comprises a set of forward primers and a set of reverse primers. The set of forward primers may consist essentially of (or consist of) forward primers complementary to IGHV4 subfamily sequences. The set of forward primers may comprise a set of external primers and a set of internal primers for nested amplification. In some embodiments, the set of forward primers for amplifying substantially all IGHV4 subfamily genes contains from 2 to 10 primers, 5 to 10 primers, or from 5 to 7 primers. In one embodiment, the set of forward primers for amplifying substantially all IGHV4 subfamily genes contains, consists essentially of, or consists of 6 primers. In another embodiment, the set of forward primers for amplifying substantially all IGHV4 subfamily genes contains, consists essentially of, or consists of 2 primers. The set of forward external primers and the set of forward internal primers together may consist essentially of 10 or 11 primers. For example, the set of forward external primers may consist of from 4 to 7 primers, and the set of forward internal primers may consist of from 4 to 7 primers. In some embodiments, the set of forward external primers consists of 5 or 6 primers, and the set of forward internal primers consists of 5 or 6 primers. In other embodiments, the set of forward external primers consists of 4 to 7 primers, and the set of forward internal primers consists of 2 primers.

In some embodiments, the set of internal and/or external forward primers targets a VH4 sequence within FR1. In some embodiments, the set of external and/or internal forward primers targets a VH4 sequence within or consisting essentially of the sequence 5′-cggaggcttcaccagtcctgggtt-3′ of IGHV4-4*01. In some embodiments, the set of forward primers is essentially as set forth in Table 1. In one embodiment, the set of forward primers consists of the six external primers VH4E1 to VH4E6 set forth in Table 1. In another embodiment, the set of forward primers consists of the five nested primers VH4N1 to VH4N5 set forth in Table 1. In another embodiment, the set of forward primers consists of the four nested primers VH4N1 to VH4N4 set forth in Table 1. In certain embodiments, the set of forward primers consists of the VH4N1 primer (5′-ggcccaggactggtgaagcct-3′, SEQ ID NO: 1) and the VH4N4 primer (5′-ggcgcaggactgttgaagcct-3′, SEQ ID NO: 2).

The primers in the forward primer set may be used in different ratios. For example, one or more primers may be used in a 2:1 to 5:1 ratio relative to the other primers in the set. In embodiments in which the forward primer set consists of six primers, three of the primers are used in a 2:1 ratio relative to the remaining three primers. In a related embodiment in which the forward primer set consists of two primers, one primer is present in twice the amount as the other primer. In one particular embodiment, the forward primer set consists of the VH4N1 primer and the VH4N4 primer, wherein the VH4N1 primer is present in twice the amount as the VH4N4 primer. In certain embodiments, the forward primers in the forward primer set are not present in equimolar amounts.

In some embodiments, the set of reverse primers consists essentially of reverse primers complementary to the 3′ end of IGHV4 subfamily sequences, or consists essentially of reverse primers complementary to IGHJ region sequences. The set of reverse primers may consist of reverse primers complementary to human IGHJ region sequences. In certain embodiments, the set of reverse primers contains from 4 to 7 primers. In one embodiment, the set of reverse primers contains, consists essentially of, or consists of 4 primers. In some embodiments, the set of reverse primers comprises a set of external primers and a set of internal primers for nested amplification. The set of reverse external primers and the set of reverse internal primers may together consist essentially of 11 primers. In some embodiments, the set of reverse external primers consists of from 4 to 7 primers, and the set of reverse internal primers consists of from 4 to 7 primers. In some embodiments, the set of reverse external primers consists of about 4 primers, and the set of reverse internal primers consists of about 7 primers.

In some embodiments, the set of external and internal reverse primers targets a J region sequence comprising, consisting essentially of, or within the sequence corresponding to 5′-ctggggccagggcaccctggtcaccgtctcctac-3′ (SEQ ID NO:3) of IGHJ1*01. The set of reverse primers may be as set forth in Table 2. In one embodiment, the set of reverse primers consists of the four external primers JHE1 to JHE4 set forth in Table 2. In another embodiment, the set of reverse primers consists of the seven nested primers JHN1 to JHN7 set forth in Table 2. Other suitable reverse primers include one or more the following:

| | Primer sequence | Germline sequence |
|---|---|---|
| Primer Name | (5′ -> 3′) | (5′ -> 3′) |
| DGX-JHE3v2 3′ | tgaagagacgg | acaatggtcac |
| Primer | tgaccattgt | cgtctcttca |
| | (SEQ ID NO: 4) | (SEQ ID NO: 5) |
| DGX-JHN3v2 3′ | acggtgaccat | caagggacaat |
| Primer | tgtcccttg | ggtcaccgt |
| | (SEQ ID NO: 6) | (SEQ ID NO: 7) |
| DGX-JHN4v2 3′ | acggtgaccag | caaggaaccct |
| Primer | ggttccttg | ggtcaccgt |
| | (SEQ ID NO: 8) | (SEQ ID NO: 9) |
| DGX-JHN7v2 3′ | acggtgaccgt | aaagggaccac |
| Primer | ggtcccttt | ggtcaccgt |
| | (SEQ ID NO: 10) | (SEQ ID NO: 11) |

These primers can be used in place of or in addition to the reverse primers set forth in Table 2. For example, JHE3v2 could be substituted for JHE3 in the external primer set in Table 2. In one embodiment, the set of reverse primers consists of the primers JHE1 (5′-tgaggagacggtgaccagggt-3′, SEQ ID NO:12), JHE2 (5′-tgaggagacagtgaccagggt-3′, SEQ ID NO:13), JHE3v2 (5′-tgaagagacggtgaccattgt-3′, SEQ ID NO:14), and JHE4 (5′-tgaggagacggtgaccgtggt-3′, SEQ ID NO:15).

The primers in the reverse primer set may also be used in different ratios. For example, one or more primers may be used in a 2:1 to 5:1 ratio relative to the other primers in the set. In embodiments in which the reverse primer set consists of four primers, two of the primers are used in a 2:1 ratio relative to the remaining two primers. In other embodiments, the reverse primers in the reverse primer set are present in equimolar amounts. For instance, in certain embodiments, the reverse primer set consists of the JHE1, JHE2, JHE3 (or JHE3v2) and JHE4 primers, wherein the primers are present in equimolar amounts. The ratios of the forward primers relative to the reverse primers may also be adjusted. For instance, in some embodiments, the forward primers are present in a higher amount than the reverse primers. In particular embodiments, one or more of the forward primers is present in a 2:1 ratio relative to the reverse primers.

In certain embodiments, the set of primers for amplifying substantially all IGHV4 subfamily genes comprises a forward set of primers consisting of 2 to 6 forward primers and a reverse set of primers consisting of 4 to 7 reverse primers. In one embodiment, the forward set of primers consists of 2 forward primers and the reverse set of primers consists of 4 reverse primers. In some embodiments, the forward primer sets may be selected from (i) VH4E1 to VH4E6, (ii) VH4N1 to VH4N4, and (iii) VH4N1 and VH4N4 and the reverse primer sets may be selected from (i) JHE1 to JHE4 or (ii) JHN1 to JHN7. In one particular embodiment, the forward set of primers consists of VH4N1 and VH4N4 and the reverse set of primers consists of JHE1, JHE2, JHE3 (or JHE3v2) and JHE4. In a related embodiment, the VH4N1 and VH4N4 forward primers are present in a 2:1 ratio relative to the JHE1, JHE2, JHE3 (or JHE3v2) and JHE4 reverse primers. In another embodiment, the VH4N1 forward primer is present in a 2:1 ratio relative to the VH4N4, JHE1, JHE2, JHE3 (or JHE3v2) and JHE4 primers.

In some embodiments, the target sequence is clonally amplified, and is at least partially sequenced. The target sequence may be amplified by any process suitable for clonal amplification, such as those using template conjugated beads or emulsions. The amplified target may be sequenced using any available sequencing technology, including Sanger sequencing, pyrosequencing, electronic DNA sequencing (e.g., pH, thermal), or other method.

In some embodiments, the sample is from a subject suspected of having an autoimmune disorder or neurological disorder, including a demyelinating disease, such as multiple sclerosis. Any B-cell or nucleic acid containing sample can be employed, such as cerebrospinal fluid, urine, or a blood sample. The blood sample may be a peripheral blood sample. Alternatively, the sample may be a DNA or RNA sample isolated from any of the forgoing.

In another aspect, the invention provides a kit for determining an antibody (e.g., IGHV4) repertoire. The kit comprises a set of non-degenerate primers, the set of non-degenerate primers comprising at least one primer specific for amplifying each of the subfamily sequences, as set forth above. The kit may contain the set of primers packaged for sale, and may further comprise computational tools (e.g., software) for classifying the patient as having, or in the early-stages of developing, an autoimmune disorder.

In another aspect, the invention provides a method and assays for evaluating a patient for the presence of an autoimmune disorder. The method comprises determining the presence of one or more amino acid substitutions in an antigen binding region of an antibody family or T-cell receptor family. For example, where the polynucleotide is an antibody gene or portion thereof, the invention evaluates the IGH-VDJ region of an antibody gene family as determined from a plurality of B cells from the patient; and classifying the patient as to an autoimmune disorder.

The invention provides improved methods to accomplish this for rearranged IGH-VDJ gene segments based on the initial work of Monson and colleagues (18) using unique combinations of non-degenerate primers, high-fidelity PCR and next-generation Sequencing (NGS). This approach to analyzing B cell repertoires also can be applied to analyzing T cell repertoires. B and T cell repertoires can be compared between different individuals or groups of individuals with different diseases to identify patterns associated with a specific disease or that differentiate different diseases. B and T cell repertoire analysis typically looks for patterns of the following types:
(1) IGH-VDJ recombinations, including the individual family and subfamilies used in the recombinations. There have been reports of IGHV family and subfamily bias in certain biological compartments, i.e. peripheral blood and CNS, and between different diseases.
(2) Frequency and/or pattern of DNA mutations, in general, at individual or groups of codons in the recombined VDJ segments.
(3) Frequency and/or pattern of DNA mutations in the recombined IGH-VDJ segments that lead to an amino acid change at each codon position in the encoded protein.

These analyses can be computational, particularly when starting with thousands of individual sequences for each individual as is the case with data generated by current NGS systems. The present invention takes this type of analysis to the next level, looking, not only at the pattern of DNA mutations that lead to amino acid changes but also the specific type of change, i.e. acidic to non-acidic or to basic and vice versa, charged to non-charged and vice versa, small to large and vice versa, hydrophilic to hydrophobic and vice versa, etc.

The specificity of a subset of antibodies produced by B cells in individuals with a given inflammatory and/or autoimmune disease is driven by exposure to specific disease-associated antigens. As such, the pattern of amino acid (aa) changes, resulting from somatic hypermutation, in the IGH-VDJ and IGL-VJ regions of antibody genes in patients with one disease will be different from those in patients with a different disease.

Although there is evidence that patterns of IGH-VDJ recombinations and the frequency and/or pattern of DNA mutations, whether or not they lead to an aa change, may differentiate patients with certain diseases with different underlying pathophysiological mechanism, these approaches may not provide sufficient diagnostic resolution to differentiate patients with closely related diseases, which is the more common clinical challenge. This is supported by a growing database of NGS-generated IGH-VDJ gene sequences for patients with MS and patients with ONDs.

Thus, in some embodiments, the invention provides a method for evaluating a patient for the presence of an autoimmune disorder, the method comprising: determining amino acid substitutions in the IGH-VDJ region of an antibody gene family from a plurality of immune cells from the patient; and classifying the patient as having or in the early-stages of developing an autoimmune disorder. The sequences may be determined using reagents (e.g. primers) as set forth herein for amplifying the IGH-VDJ region.

In some embodiments, the patient has signs or symptoms of multiple sclerosis or a autoimmune, demyelinating, or neurological condition. For example, the autoimmune disorder may be a disease causing inflammation within the central nervous system. In some embodiments, the patient has not previously been diagnosed with an autoimmune disorder.

In some embodiments, the sample is one or more of peripheral blood, urine or cerebrospinal fluid, or DNA or RNA isolated therefrom, as already described. Generally, IGH-VDJ regions from a plurality of B cells are clonally amplified, and sequenced as described. The method involves sequencing at least 50, at least 100, at least 500, at least 1000, or at least 2000 IGH-VDJ regions.

In certain embodiments, the sequenced antibody genes comprise, consist essentially of, or consist of IGHV4 family genes. The sequenced region may comprise the CDR1, CDR2, and/or CDR3. The sequenced region may further comprise IGHD and/or IGHJ sequences. In some embodiments, the sequenced region comprises or consists essentially of codons 27 to 89. For example, the sequenced region(s) may comprise one or more of codons 54, 60, 67, and 82a. In certain embodiments, the sequenced region(s) comprise one or more of codons 48, 50, 53, 58, 60, 67, 79, and 87. In certain embodiments, the sequenced region(s) comprise one or more of 33, 46, 58, 60, 65, 67, 71, 72, 78, 79, 83, 87, and 89. In still other embodiments, the sequenced region(s) comprise one or more of 32, 37, 48, 56, and 82, or one or more of 35, 47, 50, 56, 58, 63, 65, and 74. In some embodiments, the sequenced region(s) comprise one or more of 31, 37, 42, 43, 50, 62, 65, 77, 81, 82, 82, and 87.

Based upon sequence analysis, the sample is then classified as to the presence and/or type of autoimmune or neurological condition. For example, a statistically significant number of sequences may have one or more mutations selected from an S to G mutation at codon 54, an N to S mutation at codon 60, a V to L mutation at codon 67, and an S to I mutation at codon 82a, such samples being classified as having or developing relapsing remitting (RR) MS.

In some embodiments, a statistically significant number of sequences may have one or more substitutions selected from an I to V mutation at codon 48, an S to N mutation at codon 50, a Y to H mutation at codon 53, an N to K mutation at codon 58, a Y to S mutation at codon 58, an N to I or K mutation at codon 60, a T to V mutation at codon 67, an S to A mutation at codon 79, and a T to A mutation at codon 87, such samples being classified as migraine.

In some embodiments, a statistically significant number of sequences have one or more substitutions selected from a Y to F mutation at codon 33, an E to Q mutation at codon 46, an N to K mutation at codon 58, an N to S mutation at codon 58, a Y to F mutation at codon 58, an N to K mutation at codon 60, an S to R mutation at codon 65, a T to V mutation at codon 67, a V to L mutation at codon 71, a D to N mutation at codon 72, an F to V mutation at codon 78, an S to A mutation at codon 79, a T to I mutation at codon 83, a T to A mutation at codon 87, and a V to M mutation at codon 89, such samples being classified as neuromyelitis optica (NMO).

In still other embodiments, a statistically significant number of sequences may have one or more mutations selected from an N to S mutation at codon 32, an I to V mutation at codon 37, an Ito V mutation at codon 48, an S to R mutation at codon 56, and an S to R mutation at codon 82a, such samples being classified as paroxysmal nocturnal dyspnoea (PND).

In some embodiments, a statistically significant number of sequences may have one or more mutations selected from an S to G mutation at codon 35, a W to C mutation at codon 47, an E to Q mutation at codon 50, an S to I mutation at codon 56, an N to S mutation at codon 58, an L to F mutation at codon 63, an S to N mutation at codon 65, and an S to A mutation at codon 77, such samples being classified as N-sarcoid.

In some embodiments, a statistically significant number of sequences have one or more substitutions selected from an S to G mutation at codon 31, an I to V mutation at codon 37, a G to E mutation at codon 42, a K to Q mutation at codon 43, an S to C mutation at codon 53, an S to A mutation at codon 62, an S to N mutation at codon 65, a Q to K mutation at codon 77, a K to D mutation at codon 81, an L to M mutation at codon 82, an S to R mutation at codon 82, an S to G mutation at codon 82, a V to L mutation at codon 82, and a T to S mutation at codon 87, such samples being classified as systemic lupus erythematosus (N-SLE).

The sequences may be further evaluated for mutation frequency, as described in WO 2010/011894, which is hereby incorporated by reference. For example, elevated mutational frequency at one or more codons selected from 31B, 32, 40, 56, 57, 60, 81, and 89 of VH4 is suggestive of MS or probability of developing MS.

The analysis of patterns of specific amino acid changes in patients with inflammatory and/or autoimmune diseases, such as MS and related neurological diseases, will provide improved discriminatory power and can be used to develop sophisticated molecular diagnostic tools that will facilitate the early diagnosis of patients with closely related diseases.

Such analyses also enable the identification and production of agents for treating autoimmunity, by determining the sequence of an autoreactive antibody, identifying an epitope to which the autoreactive antibody binds, and synthesizing a peptide comprising the epitope. The epitope can be is identified, for example, by screening a peptide library representing the size of antibody epitopes, or alternatively screening a small molecule library.

All references cited herein, including references to US Patents and US Published Patent Applications are hereby incorporated by reference in their entireties for all purposes.

### TABLES

TABLE 1

| | Primer sequence |
|---|---|
| Primer Name | (5′ -> 3′) |
| Forward (5′) External | |
| Primer Regions | |
| DGX-VH4E1 5′ Primer | caggagtcgggcccaggact |
| | (SEQ ID NO: 16) |
| DGX-VH4E2 5′ Primer | caggagttgggcccaggact |
| | (SEQ ID NO: 17) |
| DGX-VH4E3 5′ Primer | caggagtccggctcaggact |
| | (SEQ ID NO: 18) |
| DGX-VH4E4 5′ Primer | caggactcgggcccaggact |
| | (SEQ ID NO: 19) |
| DGX-VH4E5 5′ Primer | cagcagtggggcgcaggact |
| | (SEQ ID NO: 20) |
| DGX-VH4E6 5′ Primer | caacagtggggcgcaggact |
| | (SEQ ID NO: 21) |
| Forward (5′) Nested | |
| Primer Regions | |
| DGX-VH4N1 5′ Primer | ggcccaggactggtgaagcct |
| | (SEQ ID NO: 22) |
| DGX-VH4N2 5′ Primer | ggctcaggactggtgaagcct |
| | (SEQ ID NO: 23) |
| DGX-VH4N3 5′ Primer | ggcccaggactgttgaagcct |
| | (SEQ ID NO: 24) |
| DGX-VH4N4 5′ Primer | ggcgcaggactgttgaagcct |
| | (SEQ ID NO: 25) |
| DGX-VH4N5 5′ Primer | ggcccaggactggtgaagctt |
| | (SEQ ID NO: 26) |

TABLE 2

| | Primer sequence | Germline sequence |
|---|---|---|
| Primer Name | (5′ -> 3′) | (5′ -> 3′) |
| Reverse (3′) External Primer Regions | | |
| DGX-JHE1 3′ Primer | tgaggagacggtgaccagggt | accctggtcaccgtctcctca |
| | (SEQ ID NO: 27) | (SEQ ID NO: 28) |
| DGX-JHE2 3′ Primer | tgaggagacagtgaccagggt | accctggtcactgtctcctca |
| | (SEQ ID NO: 29) | (SEQ ID NO: 30) |
| DGX-JHE3 3′ Primer | tgaagagacggtgaccattgtc | gacaatggtcaccgtctcttca |
| | (SEQ ID NO: 31) | (SEQ ID NO: 32) |
| DGX-JHE4 3′ Primer | tgaggagacggtgaccgtggt | accacggtcaccgtctcctca |
| | (SEQ ID NO: 33) | (SEQ ID NO: 34) |
| Reverse (3′) Nested Primer Regions | | |
| DGX-JHN1 3′ Primer | acggtgaccagggtgccctg | cagggcaccctggtcaccgt |
| | (SEQ ID NO: 35) | (SEQ ID NO: 36) |
| DGX-JHN2 3′ Primer | acagtgaccagggtgccacg | cgtggcaccctggtcactgt |
| | (SEQ ID NO: 37) | (SEQ ID NO: 38) |
| DGX-JHN3 3′ Primer | gacggtgaccattgtcccttg | caagggacaatggtcaccgtc |
| | (SEQ ID NO: 39) | (SEQ ID NO: 40) |
| DGX-JHN4 3′ Primer | gacggtgaccagggttccttg | caaggaaccctggtcaccgtc |
| | (SEQ ID NO: 41) | (SEQ ID NO: 42) |
| DGX-JHN5 3′ Primer | acggtgaccagggttccctg | cagggaaccctggtcaccgt |
| | (SEQ ID NO: 43) | (SEQ ID NO: 44) |
| DGX-JHN6 3′ Primer | acggtgaccgtggtcccttg | caagggaccacggtcaccgt |
| | (SEQ ID NO: 45) | (SEQ ID NO: 46) |
| DGX-JHN7 3′ Primer | acggtgaccgtggtccctttg | caaagggaccacggtcaccgt |
| | (SEQ ID NO: 47) | (SEQ ID NO: 48) |

### EXAMPLE 1

### 5′ Sequences for All IGHV4 Subfamilies and Reported Alleles

FIG. 7 is a list of all Human IGHV4 sequences listed in the IMGT database (http://www.imgt.org) as of September 2011, including confirmed polymorphisms (indicated by numbers following the asterisk). Only the nucleotide sequences for the codons that code for the first 19 amino acids (aa) in the variable gene segment after the leader sequence are displayed. This includes the 5′ end of the framework 1 (FR1) domain, which can be used to design 5′ (Forward) PCR primers for amplification of the IGH-VDJ region. These sequences were used in the analysis described above.

All sequences are compared to the sequence for IGHV4-4*01, which is used as a baseline sequence for the IGHV4 family. The amino acid sequence for IGHV4-4*01 is displayed above the IGHV4-4*01 nucleotide sequence. Sequences for the other VH4 family/subfamily members are also displayed and nucleotides that differ from the baseline sequence are highlighted in purple. The sequences for some IGHV4 subfamily members are truncated at the 5′ end as indicated below by a string of dots. Although these sequences will not be amplified by using the PCR amplification strategy proposed here, they are included here to be complete. There are a total of 77 sequences not including the 5′ truncated sequences.

### EXAMPLE 2

Unique IGHV4 Sequences for the Region Including aa1-aa19 (n=17)

FIG. 3 shows an alignment of the unique sequences for the region including amino acids 1 to 17 of IGHV4. There are a total of 17 unique sequences.

### EXAMPLE 3

### 3′ Sequences for All IGHJ Families and Reported Alleles

FIG. 5 is a list of all Human IGHJ sequences listed in the IMGT database (worldwideweb imgt.org) as of September 2011, including confirmed polymorphisms (indicated by numbers following the asterisk). This represents the region that can be used to design 3′ (Reverse) primers for the amplification of IGH-VDJ gene segments. These sequences were used in the analysis described above.

All sequences are compared to the sequence for IGHJ1*01, which is used as a baseline sequence for the IGHJ segment. Sequences for the other 5 JH family members are aligned with the baseline sequence and the nucleotides that differ from the baseline sequence are highlighted in purple. There are a total of 13 sequences.

Note: the actual primer sequences will be the reverse-complement of the sequences shown.

### EXAMPLE 4

Table 3 below is a summary of specific amino acid changes for a short region (aa 53-57) of the IGHV-D-J (VH4) region in a group of about 20 RRMS patients analyzed in one study. “% Seq” indicates the percent of all NGS sequences generated for the RRMS patient cohort that expressed the specific amino acid change.

TABLE 3

| | | | | | % |
|---|---|---|---|---|---|
| Group | Codon | From AA | To AA | Count | Seq |
| RRMS | 53 | H | A | 1 | 0.02 |
| RRMS | 53 | H | L | 189 | 2.86 |
| RRMS | 53 | H | N | 4 | 0.06 |
| RRMS | 53 | H | Q | 109 | 1.65 |
| RRMS | 53 | H | R | 460 | 6.96 |
| RRMS | 53 | H | T | 1 | 0.02 |
| RRMS | 53 | H | Y | 4 | 0.06 |
| RRMS | 53 | T | A | 59 | 0.89 |
| RRMS | 53 | T | H | 1 | 0.02 |
| RRMS | 53 | T | P | 13 | 0.20 |
| RRMS | 53 | T | S | 3 | 0.05 |
| RRMS | 53 | Y | D | 26 | 0.39 |
| RRMS | 53 | Y | F | 184 | 2.78 |
| RRMS | 53 | Y | G | 1339 | 20.26 |
| RRMS | 53 | Y | H | 242 | 3.66 |
| RRMS | 53 | Y | L | 1 | 0.02 |
| RRMS | 53 | Y | N | 313 | 4.74 |
| RRMS | 53 | Y | Q | 67 | 1.01 |
| RRMS | 53 | Y | R | 15 | 0.23 |
| RRMS | 53 | Y | S | 485 | 7.34 |
| RRMS | 54 | S | F | 1 | 0.02 |
| RRMS | 54 | S | G | 481 | 7.28 |
| RRMS | 54 | S | H | 10 | 0.15 |
| RRMS | 54 | S | I | 31 | 0.47 |
| RRMS | 54 | S | K | 1 | 0.02 |
| RRMS | 54 | S | L | 44 | 0.67 |
| RRMS | 54 | S | N | 942 | 14.25 |
| RRMS | 54 | S | R | 123 | 1.86 |
| RRMS | 54 | S | T | 1998 | 30.23 |
| RRMS | 55 | G | D | 1 | 0.02 |
| RRMS | 55 | G | E | 7 | 0.11 |
| RRMS | 55 | G | T | 473 | 7.16 |
| RRMS | 55 | G | W | 1 | 0.02 |
| RRMS | 56 | S | A | 3 | 0.05 |
| RRMS | 56 | S | D | 738 | 11.16 |
| RRMS | 56 | S | G | 212 | 3.21 |
| RRMS | 56 | S | I | 222 | 3.36 |
| RRMS | 56 | S | K | 1 | 0.02 |
| RRMS | 56 | S | N | 693 | 10.48 |
| RRMS | 56 | S | R | 147 | 2.22 |
| RRMS | 56 | S | T | 777 | 11.75 |
| RRMS | 56 | S | Y | 1 | 0.02 |
| RRMS | 57 | T | A | 252 | 3.81 |
| RRMS | 57 | T | D | 306 | 4.63 |
| RRMS | 57 | T | I | 21 | 0.32 |
| RRMS | 57 | T | N | 104 | 1.57 |
| RRMS | 57 | T | P | 307 | 4.64 |
| RRMS | 57 | T | S | 6 | 0.09 |

Table 4 below is a summary of specific amino acid changes for a short region (aa 53-57) of the IGHV-D-J (VH4) region in a group of 5 NMO patients analyzed in the same study as above. “% Seq” indicates the percent of all NGS sequences generated for the NMO patient cohort that expressed the specific amino acid change.

TABLE 4

| | | | | | % |
|---|---|---|---|---|---|
| Group | Codon | From AA | To AA | Count | Seq |
| NMO | 53 | H | P | 67 | 4.68 |
| NMO | 53 | H | Q | 7 | 0.49 |
| NMO | 53 | H | R | 421 | 29.40 |
| NMO | 53 | Y | F | 79 | 5.52 |
| NMO | 53 | Y | G | 303 | 21.16 |
| NMO | 53 | Y | H | 33 | 2.30 |
| NMO | 53 | Y | P | 9 | 0.63 |
| NMO | 53 | Y | Q | 14 | 0.98 |
| NMO | 53 | Y | R | 9 | 0.63 |
| NMO | 53 | Y | S | 148 | 10.34 |
| NMO | 54 | S | G | 41 | 2.86 |
| NMO | 54 | S | I | 45 | 3.14 |
| NMO | 54 | S | N | 50 | 3.49 |
| NMO | 54 | S | R | 33 | 2.30 |
| NMO | 54 | S | T | 427 | 29.82 |
| NMO | 55 | G | E | 1 | 0.07 |
| NMO | 55 | G | T | 430 | 30.03 |
| NMO | 55 | G | V | 1 | 0.07 |
| NMO | 56 | S | D | 50 | 3.49 |
| NMO | 56 | S | G | 30 | 2.09 |
| NMO | 56 | S | N | 6 | 0.42 |
| NMO | 56 | S | T | 51 | 3.56 |
| NMO | 57 | T | A | 54 | 3.77 |
| NMO | 57 | T | D | 3 | 0.21 |
| NMO | 57 | T | N | 43 | 3.00 |
| NMO | 57 | T | P | 99 | 6.91 |
| NMO | 57 | T | S | 2 | 0.14 |

Table 5 below is a summary of specific amino acid changes for a short region (aa 53-57) of the IGHV-D-J (VH4) region in a group of 5 migraine patients analyzed in the same study as above. “% Seq” indicates the percent of all NGS sequences generated for the migraine patient cohort that expressed the specific amino acid change.

TABLE 5

| | | | | | % |
|---|---|---|---|---|---|
| Group | Codon | From AA | To AA | Count | Seq |
| MIGRAINE | 53 | H | Q | 27 | 2.34 |
| MIGRAINE | 53 | H | R | 97 | 8.41 |
| MIGRAINE | 53 | Y | D | 2 | 0.17 |
| MIGRAINE | 53 | Y | F | 43 | 3.73 |
| MIGRAINE | 53 | Y | G | 341 | 29.55 |
| MIGRAINE | 53 | Y | H | 86 | 7.45 |
| MIGRAINE | 53 | Y | P | 16 | 1.39 |
| MIGRAINE | 53 | Y | Q | 9 | 0.78 |
| MIGRAINE | 53 | Y | R | 4 | 0.35 |
| MIGRAINE | 53 | Y | S | 157 | 13.60 |
| MIGRAINE | 54 | S | G | 38 | 3.29 |
| MIGRAINE | 54 | S | N | 69 | 5.98 |
| MIGRAINE | 54 | S | R | 69 | 5.98 |
| MIGRAINE | 54 | S | T | 524 | 45.41 |
| MIGRAINE | 55 | G | E | 1 | 0.09 |
| MIGRAINE | 55 | G | T | 101 | 8.75 |
| MIGRAINE | 56 | S | D | 57 | 4.94 |
| MIGRAINE | 56 | S | G | 28 | 2.43 |
| MIGRAINE | 56 | S | N | 73 | 6.33 |
| MIGRAINE | 56 | S | R | 5 | 0.43 |
| MIGRAINE | 56 | S | T | 52 | 4.51 |
| MIGRAINE | 57 | T | A | 127 | 11.01 |
| MIGRAINE | 57 | T | D | 7 | 0.61 |
| MIGRAINE | 57 | T | I | 1 | 0.09 |
| MIGRAINE | 57 | T | N | 3 | 0.26 |
| MIGRAINE | 57 | T | P | 141 | 12.22 |
| MIGRAINE | 57 | T | S | 19 | 1.65 |

Table 6 is a summary of specific amino acid changes for a short region (aa 53-57) of the IGHV-D-J (VH4) region in a group of 5 patients with paraneoplastic disease (PND) analyzed in the same study as above. “% Seq” indicates the percent of all NGS sequences generated for the PND patient cohort that expressed the specific amino acid change.

TABLE 6

| | | | | | % |
|---|---|---|---|---|---|
| Group | Codon | From AA | To AA | Count | Seq |
| PND | 53 | H | Q | 40 | 3.67 |
| PND | 53 | H | R | 99 | 9.07 |
| PND | 53 | T | S | 17 | 1.56 |
| PND | 53 | Y | D | 6 | 0.55 |
| PND | 53 | Y | F | 24 | 2.20 |
| PND | 53 | Y | G | 227 | 20.81 |
| PND | 53 | Y | H | 6 | 0.55 |
| PND | 53 | Y | Q | 13 | 1.19 |
| PND | 53 | Y | S | 48 | 4.40 |
| PND | 53 | Y | T | 1 | 0.09 |
| PND | 54 | S | G | 21 | 1.92 |
| PND | 54 | S | N | 253 | 23.19 |
| PND | 54 | S | R | 175 | 16.04 |
| PND | 54 | S | T | 291 | 26.67 |
| PND | 55 | G | E | 1 | 0.09 |
| PND | 55 | G | T | 70 | 6.42 |
| PND | 56 | S | D | 235 | 21.54 |
| PND | 56 | S | N | 135 | 12.37 |
| PND | 56 | S | R | 64 | 5.87 |
| PND | 56 | S | T | 51 | 4.67 |
| PND | 57 | T | A | 15 | 1.37 |
| PND | 57 | T | D | 24 | 2.20 |
| PND | 57 | T | I | 17 | 1.56 |
| PND | 57 | T | N | 38 | 3.48 |
| PND | 57 | T | P | 25 | 2.29 |

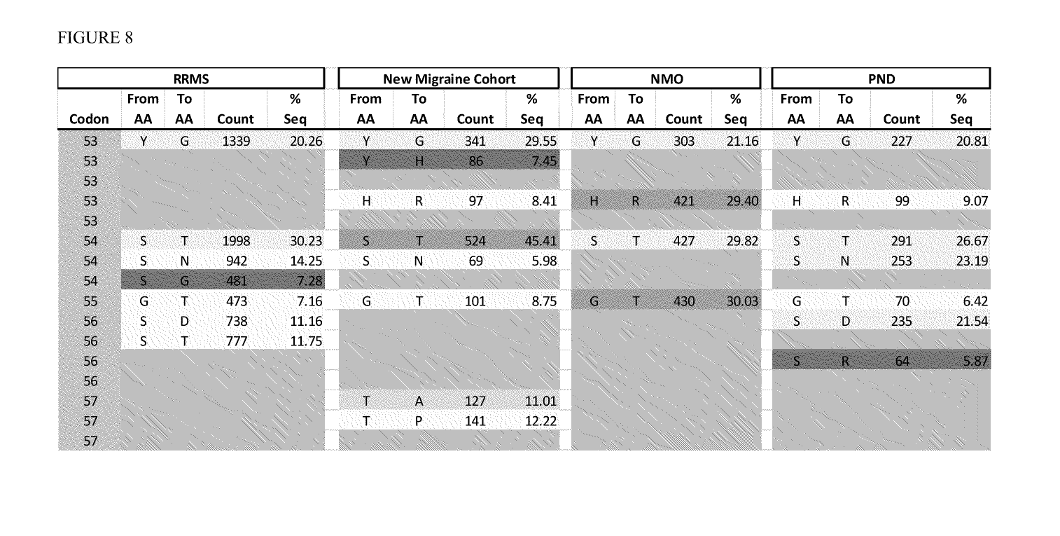

FIG. 8 shows a comparison of specific amino acid changes and the percent of all NGS sequences that express each change for a short region (aa 53-57) of the IGHV-D-J (VH4) region across the four cohorts in the same study as above.

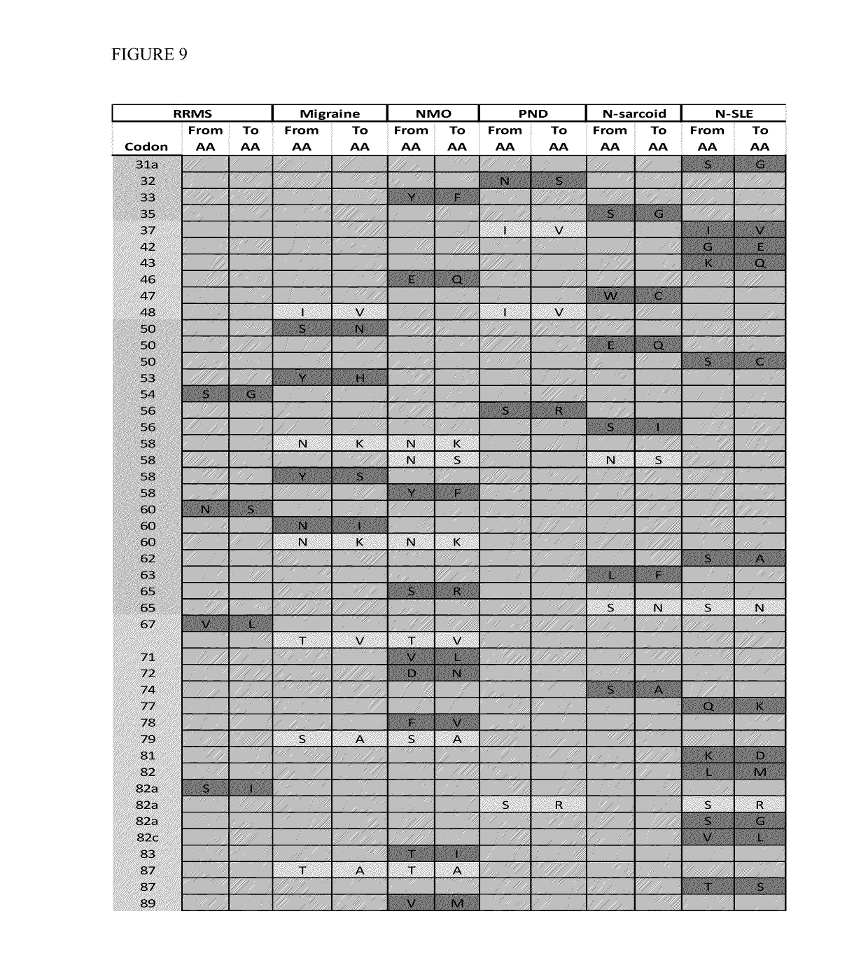

FIG. 9 shows a comparison of specific amino acid changes for almost the entire IGHV-D-J (VH4) region (aa 31-91) across the four cohorts analyzed the same study as above.

### REFERENCES:

1. Monson N L, Brezinschek H P, Brezinschek R I, Mobley A, Vaughan G K, Frohman E M, Racke M K, Lipsky P E. (2005) Receptor revision and atypical mutational characteristics in clonally expanded B cells from the cerebrospinal fluid of recently diagnosed multiple sclerosis patients. J Neuroimmunol. 158(1-2):170-81.
2. Matthews R. (2007) Autoimmune diseases. The B cell slayer. Science. 318(5854):1232-3.
3. Franciotta D, Salvetti M, Lolli F, Serafini B, Aloisi F. (2008) B cells and multiple sclerosis. Lancet Neurol. 7(9):852-8.
4. Kuenz B, Lutterotti A, Ehling R, Gneiss C, Haemmerle M, Rainer C, Deisenhammer F, Schocke M, Berger T, Reindl M. (2008) Cerebrospinal fluid B cells correlate with early brain inflammation in multiple sclerosis. PLoS One. 3(7):e2559.
5. Owens G P, Bennett J L, Gilden D H, Burgoon M P. (2006) The B cell response in multiple sclerosis. Neurol Res. 28(3):236-44.
6. Harp C, Lee J, Lambracht-Washington D, Cameron E, Olsen G, Frohman E, Racke M, Monson N. (2007) Cerebrospinal fluid B cells from multiple sclerosis patients are subject to normal germinal center selection. J Neuroimmunol. 183(1-2):189-99.
7. Vogt M H, Teunissen C E, Iacobaeus E, Heijnen D A, Breij E C, Olsson T, Brundin L, Killestein J, Dijkstra C D. (2009) Cerebrospinal fluid anti-myelin antibodies are related to magnetic resonance measures of disease activity in multiple sclerosis. J Neurol Neurosurg Psychiatry. 80(10):1110-5.
8. Breij E C, Brink B P, Veerhuis R, van den Berg C, Vloet R, Yan R, Dijkstra C D, van der Valk P, Bo L. (2008) Homogeneity of active demyelinating lesions in established multiple sclerosis. Ann Neurol. 63(1):16-25.
9. Awad A, Hemmer B, Hartung H P, Kieseier B, Bennett J L, Stuve O. (2010) Analyses of cerebrospinal fluid in the diagnosis and monitoring of multiple sclerosis. J Neuroimmunol. 219(1-2):1-7.
10. Di Noia J M, Neuberger M S. (2007) Molecular mechanisms of antibody somatic hypermutation. Annu Rev Biochem. 76:1-22.
11. http://vbase.mrc-cpe.cam.ac.uk
12. http://www.imgt.org
13. Maul R W and Gearhart P J. (2010) Controlling Somatic Hypermutation in Immunoglobulin Variable and Switch Regions. Immunol Res. 47(1-3):113-122.
14. Barrios Y, Jirholt P, Ohlin M. (2004) Length of the Antibody Heavy Chain Complementarity Determining Region 3 as a Specificity-determining factor. J Molec Recognition. 17: 332-338.
15. Tomlinson I M, Walter G, Marks J D, Llewelyn M B, Winter G. (1992) The Repertoire of Human Germline V_{H} Sequences Reveals about 50 Groups of V_{H} Segments with Different Hypervariable Loops. J Mol Biol. 227: 776-798.
16. Brezinschek H P, Brezinschek R I, Lipsky P E. (1995) Analysis of the Heavy Chain Repertoire of Human Peripheral B Cells Using Single-cell Polymerase Chain Reaction. J Immunol. 155: 190-202.
17. Wang X and Stollar B D. (2000) Human Immunoglobulin Variable Region Gene Anaysis by Single Cell RT-PCR. J Immunol Methods 244: 217-225.
18. Cameron E M, Spencer S, Lazarini J, Harp C T, Ward E S, Burgoon M, Owens G P, Racke M K, Bennett J L, Frohman E M, Monson N L. (2009) Potential of a unique antibody gene signature to predict conversion to clinically definite multiple sclerosis. J Neuroimmunol. 213(1-2):123-30.

1. A method for determining a repertoire of antibodies or T-cell receptor(s) in a patient, the method comprising:
amplifying target polynucleotides from a sample containing polynucleotides from B-cells or T-cells, the target polynucleotides encoding an antigen-binding region of an antibody gene family or T-cell receptor gene family, and said amplifying being conducted with a set of non-degenerate primers, the set of non-degenerate primers comprising at least one primer specific for amplifying each member of the family or sub-family, and
determining the sequences in the sample.

2. The method of claim 1, wherein the polynucleotides are from B-cells, and the non-degenerate primers are specific for a variable region or segment thereof

3. The method of claim 2, wherein the set of primers comprises a set of forward primers and a set of reverse primers, the set of forward primers consisting essentially of forward primers complementary to IGHV4 subfamily sequences.

4. The method of claim 3, wherein the set of forward primers consists of forward primers complementary to human IGHV4 subfamily sequences.

5. The method of any one of claims 1 to 4, wherein IGHV4 subfamily genes are set forth in FIG. 3.

6. The method of any one of claims 1 to 5, wherein the set of forward primers comprises a set of external primers and a set of internal primers for nested amplification.

7. The method of claim 5, wherein a set of forward primers for amplifying substantially all IGHV4 subfamily genes contains from 5 to 10 primers.

8. The method of claim 7, wherein a set of forward primers for amplifying substantially all IGHV4 subfamily genes contains from 5 to 7 primers.

9. The method of claim 6, wherein the set of forward external primers and the set of forward internal primers together consists essentially of 11 primers.

10. The method of claim 9, wherein the set of forward external primers consists of from 4 to 7 primers, and the set of forward internal primers consists of from 4 to 7 primers.

11. The method of claim 9, wherein the set of forward external primers consists of 5 or 6 primers, and the set of forward internal primers consists of 5 or 6 primers.

12. The method of any one of claims 6 to 11, wherein the set of internal and external forward primers targets a VH4 sequence within FR1.

13. The method of any one of claims 6 to 11, wherein the set of external and/or internal forward primers targets a VH4 sequence within or consisting essentially of the sequence 5′-cggaggcttcaccagtcctgggtt-3′ (SEQ ID NO:49) of IGHV4-4*01.

14. The method of claim 13, wherein the set of forward primers is essentially as set forth in Table 1.

15. The method of any one of claims 1 to 14, wherein the set of reverse primers consists essentially of reverse primers complementary to the 3′ end of IGHV4 subfamily sequences, or consists essentially of reverse primers complementary to IGHJ region sequences.

16. The method of claim 15, wherein the set of reverse primers consists of reverse primers complementary to human IGHJ region sequences.

17. The method of claim 15 or 16, wherein the IGHJ region sequences are as set forth in FIG. 5.

18. The method of any one of claims 15 to 17, wherein the set of reverse primers comprises a set of external primers and a set of internal primers for nested amplification.

19. The method of claim 18, wherein the set of reverse external primers and the set of reverse internal primers together consists essentially of 11 primers.

20. The method of claim 19, wherein the set of reverse external primers consists of from 4 to 7 primers, and the set of reverse internal primers consists of from 4 to 7 primers.

21. The method of claim 20, wherein the set of reverse external primers consists of about 4 primers, and the set of reverse internal primers consists of about 7 primers.

22. The method of claim 21, wherein the set of external and internal reverse primers targets a J region sequence comprising, consisting essentially of, or within the sequence corresponding to 5′-ctggggccagggcaccctggtcaccgtctcctac-3′ (SEQ ID NO:3) of IGHJ1*01.

23. The method of claim 19, wherein the set of reverse primers is essentially as set forth in Table 2.

24. The method of any one of claims 1 to 23, wherein the target sequence is clonally amplified and at least partially sequenced.

25. The method of claim 24, wherein the target sequence is amplified by emulsion PCR.

26. The method of claim 24 or 25, wherein the IGHV4 region is sequenced by Sanger sequencing or pyrosequencing.

27. The method of any one of claims 1 to 26, wherein the sample is from a subject suspected of having an autoimmune disorder.

28. The method of claim 27, wherein the autoimmune disorder is multiple sclerosis.

29. The method of any one of claims 1 to 28, wherein the sample is cerebrospinal fluid, urine or a blood sample.

30. The method of claim 29, wherein the blood sample is peripheral blood.

31. The method of any one of claims 1 to 30, further comprising identifying the presence of one or more mutations indicative of an autoimmune disease or autoimmune disease activity.

32. The method of claim 32, further comprising identifying the presence of one or more mutations indicative of MS or MS disease activity.

33. The method of claim 31 or 32, wherein the position of the one or more mutations are at codons 31B, 32, 40, 56, 57, 60, 81, and 89.

34. The method of any one of claims 31 to 33, further comprising, determining the frequency of said one or more mutations in the sample.

35. A kit for determining a IGHV4 repertoire, the kit comprising:
a set of non-degenerate primers, the set of non-degenerate primers comprising at least one primer specific for amplifying each of the IGHV4 subfamily sequences.

36. The kit of claim 35, wherein the external forward primers and/or external reverse primers consist of from 5 to 7 primers for amplifying substantially all IGHV4 sequences.

37. The kit of claim 36, wherein the set of primers comprises a set of forward primers and a set of reverse primers, the set of forward primers consisting essentially of forward primers complementary to IGHV4 subfamily sequences and the set of reverse primers consisting essentially of reverse primers complementary to IGHJ family sequences.

38. The kit of claim 36 or 37, wherein the set of forward primers consists of forward primers complementary to human IGHV4 subfamily sequences shown in Example 3.

39. The kit of claim 35, wherein the set of forward primers comprises a set of external primers and a set of internal primers for nested amplification.

40. The kit of claim 35, wherein a set of external forward primers for amplifying substantially all IGHV4 subfamily genes contains from 5 to 10 primers.

41. The kit of claim 36, wherein the set of forward external primers and the set of forward internal primers together consists essentially of 11 primers.

42. The kit of claim 40 or 41, wherein the set of forward external primers consists of from 4 to 7 primers, and the set of forward internal primers consists of from 4 to 7 primers.

43. The kit of claim 42, wherein the set of forward external primers consists of 5 or 6 primers, and the set of forward internal primers consists of 5 or 6 primers.

41. The kit of claim 38, wherein the set of external and internal forward primers targets a VH4 sequence comprising, consisting essentially of, or within the sequence corresponding to 5′-cggaggcttcaccagtcctgggtt-3′ (SEQ ID NO:49) of IGHV4-4*01.

42. The kit of claim 41, wherein the set of forward external and/or internal primers is essentially as set forth in Table 1.

43. The kit of any one of claims 35 to 42, wherein the set of reverse primers consists essentially of reverse primers complementary to the 3′ end of IGHV4 subfamily sequences, or consists essentially of reverse primers complementary to IGHJ region sequences.

44. The kit of claim 43, wherein the set of reverse primers consists of reverse primers complementary to human IGHJ region sequences.

45. The kit of claim 44, wherein the IGHJ region sequences are set forth in Table 2.

46. The kit of any one of claims 43 to 45, wherein the set of reverse primers comprises a set of external primers and a set of internal primers for nested amplification.

47. The kit of claim 46, wherein the set of reverse external primers and the set of reverse internal primers together consists essentially of 11 primers.

48. The kit of claim 47, wherein the set of reverse external primers consists of from 4 to 7 primers, and the set of reverse internal primers consists of from 4 to 7 primers.

49. The kit of claim 48, wherein the set of reverse external primers consists of about 4 primers, and the set of reverse internal primers consists of about 7 primers.

50. The kit of claim 46, wherein the set of external and/or internal reverse primers targets a IGHJ region sequence comprising, consisting essentially of, or within the sequence corresponding to 5′-ctggggccagggcaccctggtcaccgtctcctac-3′ (SEQ ID NO:3) of IGHJ1*01.

51. The kit of claim 50, wherein the set of reverse primers is essentially as set forth in Table 2.

52. The kit of any one of claims 35 to 51, wherein the set of primers are packaged for sale.

53. A kit for evaluating human biofluids comprising reagents for determining amino acid substitutions in the IGH-VDJ region of an antibody gene family from a plurality of immune cells from a patient; and computational tools for classifying the patient as having, or in the early-stages of developing, an autoimmune disorder.

54. The kit of claim 53, wherein the reagents include primers as set forth in any one of claims 35 to 52 for amplifying the IGH-VDJ region.

55. A method for evaluating a patient for the presence of an autoimmune disorder, the method comprising: determining amino acid substitutions in the IGH-VDJ region of an antibody gene family from a plurality of immune cells from the patient; and classifying the patient as having or in the early-stages of developing an autoimmune disorder.

56. The method of claim 55, wherein reagents include primers as set forth in any one of claims 35 to 52 for amplifying the IGH-VDJ region.

57. The method of claim 55, wherein the patient has signs or symptoms of multiple sclerosis or a demyelinating condition.

58. The method of claim 55, wherein the autoimmune disorder comprises disease causing inflammation within the central nervous system.

59. The method of claim 55, wherein the patient has not previously been diagnosed with an autoimmune disorder.

60. The method of any one of claims 55 to 59, wherein the sample is one or more of peripheral blood, urine or cerebrospinal fluid.

61. The method of any one of claims 55 to 60, wherein IGH-VDJ regions from a plurality of B cells are clonally amplified, and sequenced.

62. The method of claim 61, wherein the IGH-VDJ regions are amplified and sequenced according to one or more of claims 1 to 34, or a kit according to claims 35 to 54.

63. The method of claim 56, wherein the antibody genes comprise, consist essentially of, or consist of IGHV4 family genes.

64. The method of claim 57, wherein the sequenced region comprises CDR1, CDR2, and/or CDR3.

65. The method of claim 64, wherein the sequenced region comprises IGHD and/or IGHJ sequences.

66. The method of claim 64, wherein the sequenced region comprises CDR3.

67. The method of any one of claims 63 to 65, wherein the sequenced region comprises or consists essentially of codons 27 to 89.

68. The method of claim 67, wherein the sequenced region comprises one or more of codons 54, 60, 67, and 82a.

69. The method of claim 67, wherein the sequenced region comprises one or more of codons 48, 50, 53, 58, 60, 67, 79, and 87.

70. The method of claim 67, wherein the sequenced region comprises one or more of 33, 46, 58, 60, 65, 67, 71, 72, 78, 79, 83, 87, and 89.

71. The method of claim 67, wherein the sequenced region comprises one or more of 32, 37, 48, 56, and 82.

72. The method of claim 67, wherein the sequenced region comprises one or more of 35, 47, 50, 56, 58, 63, 65, and 74.

73. The method of claim 67, wherein the sequenced region comprises one or more of 31, 37, 42, 43, 50, 62, 65, 77, 81, 82, 82, and 87.

74. The method of any one of claims 55 to 73, wherein the sample is classified based upon information from Appendix 4.

75. The method of claim 74, wherein a statistically significant number of sequences have one or more mutations selected from an S to G mutation at codon 54, an N to S mutation at codon 60, a V to L mutation at codon 67, and an S to I mutation at codon 82a.

76. The method of claim 74, wherein a statistically significant number of sequences have one or more substitutions selected from an I to V mutation at codon 48, an S to N mutation at codon 50, a Y to H mutation at codon 53, an N to K mutation at codon 58, a Y to S mutation at codon 58, an N to I or K mutation at codon 60, a T to V mutation at codon 67, an S to A mutation at codon 79, and a T to A mutation at codon 87.

77. The method of claim 74, wherein a statistically significant number of sequences have one or more substitutions selected from a Y to F mutation at codon 33, an E to Q mutation at codon 46, an N to K mutation at codon 58, an N to S mutation at codon 58, a Y to F mutation at codon 58, an N to K mutation at codon 60, an S to R mutation at codon 65, a T to V mutation at codon 67, a V to L mutation at codon 71, a D to N mutation at codon 72, an F to V mutation at codon 78, an S to A mutation at codon 79, a T to I mutation at codon 83, a T to A mutation at codon 87, and a V to M mutation at codon 89.

78. The method of claim 74, wherein a statistically significant number of sequences have one or more mutations selected from an N to S mutation at codon 32, an I to V mutation at codon 37, an I to V mutation at codon 48, an S to R mutation at codon 56, and an S to R mutation at codon 82a.

79. The method of claim 74, wherein a statistically significant number of sequences have one or more mutations selected from an S to G mutation at codon 35, a W to C mutation at codon 47, an E to Q mutation at codon 50, an S to I mutation at codon 56, an N to S mutation at codon 58, an L to F mutation at codon 63, an S to N mutation at codon 65, and an S to A mutation at codon 77.

80. The method of claim 74, wherein a statistically significant number of sequences have one or more substitutions selected from an S to G mutation at codon 31, an I to V mutation at codon 37, a G to E mutation at codon 42, a K to Q mutation at codon 43, an S to C mutation at codon 53, an S to A mutation at codon 62, an S to N mutation at codon 65, a Q to K mutation at codon 77, a K to D mutation at codon 81, an L to M mutation at codon 82, an S to R mutation at codon 82, an S to G mutation at codon 82, a V to L mutation at codon 82, and a T to S mutation at codon 87.

81. The method of any one of claims 55 to 80, wherein the sample is further evaluated for mutation frequency.

82. The method of any one of claims 55 to 81, wherein the patient is classified as one of RRMS, migraine, NMO, PND, N-sarcoid, or N-SLE.

83. A method for making an agent for treating autoimmunity, comprising, determining the sequence of an autoreactive antibody, optionally in accordance with one or more of claims 1 to 34, identifying an epitope to which said autoreactive antibody binds, and synthesizing a peptide comprising said epitope to thereby make an agent for treating autoimmunity.

84. The method of claim 83, wherein said epitope is identified by screening a peptide or small molecule library.

85. An agent produced by the method of claim 83 or 84.


SEQUENCE LISTING (170 sequences)

SEQ ID NO: 1 - DNA, 21 bp, Artificial Sequence
  VH4N1 primer
ggcccaggactggtgaagcct

SEQ ID NO: 2 - DNA, 21 bp, Artificial Sequence
  VH4N4 primer
ggcgcaggactgttgaagcct

SEQ ID NO: 3 - DNA, 34 bp, Homo sapiens
ctggggccagggcaccctggtcaccgtctcctac

SEQ ID NO: 4 - DNA, 21 bp, Artificial Sequence
  DGX-JHE3v2 3′ Primer
tgaagagacggtgaccattgt

SEQ ID NO: 5 - DNA, 21 bp, Homo sapiens
acaatggtcaccgtctcttca

SEQ ID NO: 6 - DNA, 20 bp, Artificial Sequence
  DGX-JHN3v2 3′ Primer
acggtgaccattgtcccttg

SEQ ID NO: 7 - DNA, 20 bp, Homo sapiens
caagggacaatggtcaccgt

SEQ ID NO: 8 - DNA, 20 bp, Artificial Sequence
  DGX-JHN4v2 3′ Primer
acggtgaccagggttccttg

SEQ ID NO: 9 - DNA, 20 bp, Homo sapiens
caaggaaccctggtcaccgt

SEQ ID NO: 10 - DNA, 20 bp, Artificial Sequence
  DGX-JHN7v2 3′ Primer
acggtgaccgtggtcccttt

SEQ ID NO: 11 - DNA, 20 bp, Homo sapiens
aaagggaccacggtcaccgt

SEQ ID NO: 12 - DNA, 21 bp, Artificial Sequence
  JHE1 primer
tgaggagacggtgaccagggt

SEQ ID NO: 13 - DNA, 21 bp, Artificial Sequence
  JHE2 primer
tgaggagacagtgaccagggt

SEQ ID NO: 14 - DNA, 21 bp, Artificial Sequence
  JHE3v2 primer
tgaagagacggtgaccattgt

SEQ ID NO: 15 - DNA, 21 bp, Artificial Sequence
  JHE4 primer
tgaggagacggtgaccgtggt

SEQ ID NO: 16 - DNA, 20 bp, Artificial Sequence
  DGX-VH4E1 5′ Primer
caggagtcgggcccaggact

SEQ ID NO: 17 - DNA, 20 bp, Artificial Sequence
  DGX-VH4E2 5′ Primer
caggagttgggcccaggact

SEQ ID NO: 18 - DNA, 20 bp, Artificial Sequence
  DGX-VH4E3 5′ Primer
caggagtccggctcaggact

SEQ ID NO: 19 - DNA, 20 bp, Artificial Sequence
  DGX-VH4E4 5′ Primer
caggactcgggcccaggact

SEQ ID NO: 20 - DNA, 20 bp, Artificial Sequence
  DGX-VH4E5 5′ Primer
cagcagtggggcgcaggact

SEQ ID NO: 21 - DNA, 20 bp, Artificial Sequence
  DGX-VH4E6 5′ Primer
caacagtggggcgcaggact

SEQ ID NO: 22 - DNA, 21 bp, Artificial Sequence
  DGX-VH4N1 5′ Primer
ggcccaggactggtgaagcct

SEQ ID NO: 23 - DNA, 21 bp, Artificial Sequence
  DGX-VH4N2 5′ Primer
ggctcaggactggtgaagcct

SEQ ID NO: 24 - DNA, 21 bp, Artificial Sequence
  DGX-VH4N3 5′ Primer
ggcccaggactgttgaagcct

SEQ ID NO: 25 - DNA, 21 bp, Artificial Sequence
  DGX-VH4N4 5′ Primer
ggcgcaggactgttgaagcct

SEQ ID NO: 26 - DNA, 21 bp, Artificial Sequence
  DGX-VH4N5 5′ Primer
ggcccaggactggtgaagctt

SEQ ID NO: 27 - DNA, 21 bp, Artificial Sequence
  DGX-JHE1 3′ Primer
tgaggagacggtgaccagggt

SEQ ID NO: 28 - DNA, 21 bp, Homo sapiens
accctggtcaccgtctcctca

SEQ ID NO: 29 - DNA, 21 bp, Artificial Sequence
  DGX-JHE2 3′ Primer
tgaggagacagtgaccagggt

SEQ ID NO: 30 - DNA, 21 bp, Homo sapiens
accctggtcactgtctcctca

SEQ ID NO: 31 - DNA, 22 bp, Artificial Sequence
  DGX-JHE3 3′ Primer
tgaagagacggtgaccattgtc

SEQ ID NO: 32 - DNA, 22 bp, Homo sapiens
gacaatggtcaccgtctcttca

SEQ ID NO: 33 - DNA, 21 bp, Artificial Sequence
  DGX-JHE4 3′ Primer
tgaggagacggtgaccgtggt

SEQ ID NO: 34 - DNA, 21 bp, Homo sapiens
accacggtcaccgtctcctca

SEQ ID NO: 35 - DNA, 20 bp, Artificial Sequence
  DGX-JHN1 3′ Primer
acggtgaccagggtgccctg

SEQ ID NO: 36 - DNA, 20 bp, Homo sapiens
cagggcaccctggtcaccgt

SEQ ID NO: 37 - DNA, 20 bp, Artificial Sequence
  DGX-JHN2 3′ Primer
acagtgaccagggtgccacg

SEQ ID NO: 38 - DNA, 20 bp, Homo sapiens
cgtggcaccctggtcactgt

SEQ ID NO: 39 - DNA, 21 bp, Artificial Sequence
  DGX-JHN3 3′ Primer
gacggtgaccattgtcccttg

SEQ ID NO: 40 - DNA, 21 bp, Homo sapiens
caagggacaatggtcaccgtc

SEQ ID NO: 41 - DNA, 21 bp, Artificial Sequence
  DGX-JHN4 3′ Primer
gacggtgaccagggttccttg

SEQ ID NO: 42 - DNA, 21 bp, Homo sapiens
caaggaaccctggtcaccgtc

SEQ ID NO: 43 - DNA, 20 bp, Artificial Sequence
  DGX-JHN5 3′ Primer
acggtgaccagggttccctg

SEQ ID NO: 44 - DNA, 20 bp, Homo sapiens
cagggaaccctggtcaccgt

SEQ ID NO: 45 - DNA, 20 bp, Artificial Sequence
  DGX-JHN6 3′ Primer
acggtgaccgtggtcccttg

SEQ ID NO: 46 - DNA, 20 bp, Homo sapiens
caagggaccacggtcaccgt

SEQ ID NO: 47 - DNA, 21 bp, Artificial Sequence
  DGX-JHN7 3′ Primer
acggtgaccgtggtccctttg

SEQ ID NO: 48 - DNA, 21 bp, Homo sapiens
caaagggaccacggtcaccgt

SEQ ID NO: 49 - DNA, 24 bp, Homo sapiens
cggaggcttcaccagtcctgggtt

SEQ ID NO: 50 - protein, 19 aa, Homo sapiens
QVQLQESGPGLVKPSGTLS

SEQ ID NO: 51 - DNA, 57 bp, Homo sapiens
caggtgcagctgcaggagtcgggcccaggactggtgaagcctccggggaccctgtcc

SEQ ID NO: 52 - DNA, 57 bp, Homo sapiens
caggtgcagctgcaggagtcgggcccaggactggtgaagccttcggggaccctgtcc

SEQ ID NO: 53 - DNA, 57 bp, Homo sapiens
caggtgcagctgcaggagttgggcccaggactggtgaagcctccggggaccctgtcc

SEQ ID NO: 54 - DNA, 57 bp, Homo sapiens
caggtgcagctgcaggagtcgggcccaggactggtgaagccttcggagaccctgtcc

SEQ ID NO: 55 - DNA, 57 bp, Homo sapiens
caggtgcagctgcaggagtcgggcccaggactggtgaagccttcggacaccctgtcc

SEQ ID NO: 56 - DNA, 57 bp, Homo sapiens
caggtgcagctgcaggagtcgggcccaggactggtgaagccttcacagaccctgtcc

SEQ ID NO: 57 - DNA, 57 bp, Homo sapiens
caggtgcagctacaggagtcgggcccaggactggtgaagccttcggacaccctgtcc

SEQ ID NO: 58 - DNA, 57 bp, Homo sapiens
cagctgcagctgcaggagtccggctcaggactggtgaagccttcacagaccctgtcc

SEQ ID NO: 59 - DNA, 57 bp, Homo sapiens
caggtgcagctgcaggactcgggcccaggactggtgaagccttcacagaccctgtcc

SEQ ID NO: 60 - DNA, 57 bp, Homo sapiens
caggtgcggctgcaggagtcgggcccaggactggtgaagccttcacagaccctgtcc

SEQ ID NO: 61 - DNA, 57 bp, Homo sapiens
caggtgcagctgcaggagtcgggcccaggactgttgaagccttcacagaccctgtcc

SEQ ID NO: 62 - DNA, 57 bp, Homo sapiens
caggtgcagctacagcagtggggcgcaggactgttgaagccttcggagaccctgtcc

SEQ ID NO: 63 - DNA, 57 bp, Homo sapiens
caggtgcagctacaacagtggggcgcaggactgttgaagccttcggagaccctgtcc

SEQ ID NO: 64 - DNA, 57 bp, Homo sapiens
cagctgcagctgcaggagtcgggcccaggactggtgaagccttcggagaccctgtcc

SEQ ID NO: 65 - DNA, 57 bp, Homo sapiens
cagctgcagctgcaggagtcgggcccaggactggtgaagccttcggagaccccgtcc

SEQ ID NO: 66 - DNA, 57 bp, Homo sapiens
cggctgcagctgcaggagtcgggcccaggactggtgaagccttcggagaccctgtcc

SEQ ID NO: 67 - DNA, 57 bp, Homo sapiens
caggtgcagctgcaggagtcgggcccaggactggtgaagctttcggagaccctgtcc

SEQ ID NO: 68 - DNA, 20 bp, Artificial Sequence
  IGHV-FR1 5′ Forward Primers
caggagtcgggcccaggact

SEQ ID NO: 69 - DNA, 20 bp, Artificial Sequence
  IGHV-FR1 5′ Forward Primers
caggagttgggcccaggact

SEQ ID NO: 70 - DNA, 20 bp, Artificial Sequence
  IGHV-FR1 5′ Forward Primers
caggagtccggctcaggact

SEQ ID NO: 71 - DNA, 20 bp, Artificial Sequence
  IGHV-FR1 5′ Forward Primers
caggactcgggcccaggact

SEQ ID NO: 72 - DNA, 20 bp, Artificial Sequence
  IGHV-FR1 5′ Forward Primers
cagcagtggggcgcaggact

SEQ ID NO: 73 - DNA, 20 bp, Artificial Sequence
  IGHV-FR1 5′ Forward Primers
caacagtggggcgcaggact

SEQ ID NO: 74 - DNA, 52 bp, Homo sapiens
gctgaatacttccagcactggggccagggcaccctggtcaccgtctcctcag

SEQ ID NO: 75 - DNA, 52 bp, Homo sapiens
tactggtacttcgatctctggggccgtggcaccctggtcactgtctcctcag

SEQ ID NO: 76 - DNA, 50 bp, Homo sapiens
tgatgcttttgatgtctggggccaagggacaatggtcaccgtctcttcag

SEQ ID NO: 77 - DNA, 50 bp, Homo sapiens
tgatgcttttgatatctggggccaagggacaatggtcaccgtctcttcag

SEQ ID NO: 78 - DNA, 48 bp, Homo sapiens
actactttgactactggggccaaggaaccctggtcaccgtctcctcag

SEQ ID NO: 79 - DNA, 48 bp, Homo sapiens
actactttgactactggggccagggaaccctggtcaccgtctcctcag

SEQ ID NO: 80 - DNA, 48 bp, Homo sapiens
gctactttgactactggggccaagggaccctggtcaccgtctcctcag

SEQ ID NO: 81 - DNA, 51 bp, Homo sapiens
acaactggttcgactcctggggccaaggaaccctggtcaccgtctcctcag

SEQ ID NO: 82 - DNA, 51 bp, Homo sapiens
acaactggttcgacccctggggccagggaaccctggtcaccgtctcctcag

SEQ ID NO: 83 - DNA, 52 bp, Homo sapiens
tactacggtatggacgtctgggggcaagggaccacggtcaccgtctcctcag

SEQ ID NO: 84 - DNA, 51 bp, Homo sapiens
tactacggtatggacgtctggggccaagggaccacggtcaccgtctcctca

SEQ ID NO: 85 - DNA, 51 bp, Homo sapiens
tactactacatggacgtctggggcaaagggaccacggtcaccgtctcctca

SEQ ID NO: 86 - DNA, 52 bp, Homo sapiens
tactacggtatggacgtctggggcaaagggaccacggtcaccgtctcctcag

SEQ ID NO: 87 - DNA, 20 bp, Artificial Sequence
  IGHJ 3′ Reverse Primers
cagggcaccctggtcaccgt

SEQ ID NO: 88 - DNA, 20 bp, Artificial Sequence
  IGHJ 3′ Reverse Primers
cgtggcaccctggtcactgt

SEQ ID NO: 89 - DNA, 20 bp, Artificial Sequence
  IGHJ 3′ Reverse Primers
caagggacaatggtcaccgt

SEQ ID NO: 90 - DNA, 20 bp, Artificial Sequence
  IGHJ 3′ Reverse Primers
caaggaaccctggtcaccgt

SEQ ID NO: 91 - DNA, 20 bp, Artificial Sequence
  IGHJ 3′ Reverse Primers
cagggaaccctggtcaccgt

SEQ ID NO: 92 - DNA, 20 bp, Artificial Sequence
  IGHJ 3′ Reverse Primers
caagggaccacggtcaccgt

SEQ ID NO: 93 - DNA, 20 bp, Artificial Sequence
  IGHJ 3′ Reverse Primers
aaagggaccacggtcaccgt

SEQ ID NO: 94 - DNA, 57 bp, Homo sapiens; identical to SEQ ID NO: 51

SEQ ID NO: 95 - DNA, 57 bp, Homo sapiens; identical to SEQ ID NO: 52

SEQ ID NO: 96 - DNA, 57 bp, Homo sapiens; identical to SEQ ID NO: 51

SEQ ID NO: 97 - DNA, 57 bp, Homo sapiens; identical to SEQ ID NO: 51

SEQ ID NO: 98 - DNA, 57 bp, Homo sapiens; identical to SEQ ID NO: 53

SEQ ID NO: 99 - DNA, 57 bp, Homo sapiens; identical to SEQ ID NO: 54

SEQ ID NO: 100 - DNA, 57 bp, Homo sapiens; identical to SEQ ID NO: 55

SEQ ID NO: 101 - DNA, 57 bp, Homo sapiens; identical to SEQ ID NO: 56

SEQ ID NO: 102 - DNA, 57 bp, Homo sapiens; identical to SEQ ID NO: 55

SEQ ID NO: 103 - DNA, 57 bp, Homo sapiens; identical to SEQ ID NO: 55

SEQ ID NO: 104 - DNA, 57 bp, Homo sapiens; identical to SEQ ID NO: 55

SEQ ID NO: 105 - DNA, 57 bp, Homo sapiens; identical to SEQ ID NO: 57

SEQ ID NO: 106 - DNA, 57 bp, Homo sapiens; identical to SEQ ID NO: 58

SEQ ID NO: 107 - DNA, 57 bp, Homo sapiens; identical to SEQ ID NO: 58

SEQ ID NO: 108 - DNA, 57 bp, Homo sapiens; identical to SEQ ID NO: 58

SEQ ID NO: 109 - DNA, 57 bp, Homo sapiens; identical to SEQ ID NO: 58

SEQ ID NO: 110 - DNA, 57 bp, Homo sapiens; identical to SEQ ID NO: 56

SEQ ID NO: 111 - DNA, 57 bp, Homo sapiens; identical to SEQ ID NO: 55

SEQ ID NO: 112 - DNA, 57 bp, Homo sapiens; identical to SEQ ID NO: 56

SEQ ID NO: 113 - DNA, 57 bp, Homo sapiens; identical to SEQ ID NO: 59

SEQ ID NO: 114 - DNA, 57 bp, Homo sapiens; identical to SEQ ID NO: 56

SEQ ID NO: 115 - DNA, 57 bp, Homo sapiens; identical to SEQ ID NO: 56

SEQ ID NO: 116 - DNA, 57 bp, Homo sapiens; identical to SEQ ID NO: 56

SEQ ID NO: 117 - DNA, 57 bp, Homo sapiens; identical to SEQ ID NO: 60

SEQ ID NO: 118 - DNA, 57 bp, Homo sapiens; identical to SEQ ID NO: 56

SEQ ID NO: 119 - DNA, 57 bp, Homo sapiens; identical to SEQ ID NO: 56

SEQ ID NO: 120 - DNA, 57 bp, Homo sapiens; identical to SEQ ID NO: 56

SEQ ID NO: 121 - DNA, 57 bp, Homo sapiens; identical to SEQ ID NO: 56

SEQ ID NO: 122 - DNA, 57 bp, Homo sapiens; identical to SEQ ID NO: 56

SEQ ID NO: 123 - DNA, 57 bp, Homo sapiens; identical to SEQ ID NO: 61

SEQ ID NO: 124 - DNA, 57 bp, Homo sapiens; identical to SEQ ID NO: 62

SEQ ID NO: 125 - DNA, 57 bp, Homo sapiens; identical to SEQ ID NO: 63

SEQ ID NO: 126 - DNA, 57 bp, Homo sapiens; identical to SEQ ID NO: 62

SEQ ID NO: 127 - DNA, 57 bp, Homo sapiens; identical to SEQ ID NO: 62

SEQ ID NO: 128 - DNA, 57 bp, Homo sapiens; identical to SEQ ID NO: 62

SEQ ID NO: 129 - DNA, 57 bp, Homo sapiens; identical to SEQ ID NO: 62

SEQ ID NO: 130 - DNA, 57 bp, Homo sapiens; identical to SEQ ID NO: 62

SEQ ID NO: 131 - DNA, 57 bp, Homo sapiens; identical to SEQ ID NO: 62

SEQ ID NO: 132 - DNA, 57 bp, Homo sapiens; identical to SEQ ID NO: 56

SEQ ID NO: 133 - DNA, 57 bp, Homo sapiens; identical to SEQ ID NO: 54

SEQ ID NO: 134 - DNA, 57 bp, Homo sapiens; identical to SEQ ID NO: 62

SEQ ID NO: 135 - DNA, 57 bp, Homo sapiens; identical to SEQ ID NO: 62

SEQ ID NO: 136 - DNA, 57 bp, Homo sapiens; identical to SEQ ID NO: 64

SEQ ID NO: 137 - DNA, 57 bp, Homo sapiens; identical to SEQ ID NO: 64

SEQ ID NO: 138 - DNA, 57 bp, Homo sapiens; identical to SEQ ID NO: 64

SEQ ID NO: 139 - DNA, 57 bp, Homo sapiens; identical to SEQ ID NO: 65

SEQ ID NO: 140 - DNA, 57 bp, Homo sapiens; identical to SEQ ID NO: 66

SEQ ID NO: 141 - DNA, 57 bp, Homo sapiens; identical to SEQ ID NO: 64

SEQ ID NO: 142 - DNA, 57 bp, Homo sapiens; identical to SEQ ID NO: 54

SEQ ID NO: 143 - DNA, 57 bp, Homo sapiens; identical to SEQ ID NO: 54

SEQ ID NO: 144 - DNA, 57 bp, Homo sapiens; identical to SEQ ID NO: 54

SEQ ID NO: 145 - DNA, 57 bp, Homo sapiens; identical to SEQ ID NO: 67

SEQ ID NO: 146 - DNA, 57 bp, Homo sapiens; identical to SEQ ID NO: 67

SEQ ID NO: 147 - DNA, 57 bp, Homo sapiens; identical to SEQ ID NO: 54

SEQ ID NO: 148 - DNA, 57 bp, Homo sapiens; identical to SEQ ID NO: 54

SEQ ID NO: 149 - DNA, 57 bp, Homo sapiens; identical to SEQ ID NO: 54

SEQ ID NO: 150 - DNA, 57 bp, Homo sapiens; identical to SEQ ID NO: 54

SEQ ID NO: 151 - DNA, 57 bp, Homo sapiens; identical to SEQ ID NO: 54

SEQ ID NO: 152 - DNA, 57 bp, Homo sapiens; identical to SEQ ID NO: 54

SEQ ID NO: 153 - DNA, 57 bp, Homo sapiens; identical to SEQ ID NO: 54

SEQ ID NO: 154 - DNA, 57 bp, Homo sapiens; identical to SEQ ID NO: 54

SEQ ID NO: 155 - DNA, 57 bp, Homo sapiens; identical to SEQ ID NO: 54

SEQ ID NO: 156 - DNA, 57 bp, Homo sapiens; identical to SEQ ID NO: 54

SEQ ID NO: 157 - DNA, 57 bp, Homo sapiens; identical to SEQ ID NO: 55

SEQ ID NO: 158 - DNA, 57 bp, Homo sapiens; identical to SEQ ID NO: 54

SEQ ID NO: 159 - DNA, 57 bp, Homo sapiens; identical to SEQ ID NO: 62

SEQ ID NO: 160 - DNA, 57 bp, Homo sapiens; identical to SEQ ID NO: 54

SEQ ID NO: 161 - DNA, 57 bp, Homo sapiens; identical to SEQ ID NO: 56

SEQ ID NO: 162 - DNA, 57 bp, Homo sapiens; identical to SEQ ID NO: 54

SEQ ID NO: 163 - DNA, 57 bp, Homo sapiens; identical to SEQ ID NO: 54

SEQ ID NO: 164 - DNA, 57 bp, Homo sapiens; identical to SEQ ID NO: 64

SEQ ID NO: 165 - DNA, 57 bp, Homo sapiens; identical to SEQ ID NO: 54

SEQ ID NO: 166 - DNA, 57 bp, Homo sapiens; identical to SEQ ID NO: 54

SEQ ID NO: 167 - DNA, 57 bp, Homo sapiens; identical to SEQ ID NO: 54

SEQ ID NO: 168 - DNA, 57 bp, Homo sapiens; identical to SEQ ID NO: 54

SEQ ID NO: 169 - DNA, 57 bp, Homo sapiens; identical to SEQ ID NO: 54

SEQ ID NO: 170 - DNA, 57 bp, Homo sapiens; identical to SEQ ID NO: 54